(19) United States
(12) Patent Application Publication
(10) Pub. No.: US 2016/0237511 A1
(43) Pub. Date: Aug. 18, 2016

(54) METHODS FOR DETERMINING VIRAL SENSITIVITY TO VIRAL INHIBITORS

(71) Applicant: Laboratory Corporation of America Holdings, Burlington, NC (US)

(72) Inventor: Jacqueline Denise Reeves, San Francisco, CA (US)

(73) Assignee: Laboratory Corporation of America Holdings, Burlington, NC (US)

(21) Appl. No.: 15/040,480

(22) Filed: Feb. 10, 2016

Related U.S. Application Data
(62) Division of application No. 14/217,410, filed on Mar. 17, 2014, now abandoned.
(60) Provisional application No. 61/802,212, filed on Mar. 15, 2013.
(60) Provisional application No. 61/831,134, filed on Jun. 4, 2013.
(60) Provisional application No. 61/839,947, filed on Jun. 27, 2013.

(51) Int. Cl. C12Q 1/70; C12Q 1/68

(52) U.S. Cl. CPC C12Q 1/707; C12Q 1/6897; C12Q 2600/106

(57) ABSTRACT

Methods and compositions for the efficient and accurate determination of susceptibility of a hepatitis C virus (HCV) or HCV population to an HCV inhibitor. The inhibitor may include, for example, an interferon (IFN), ribavirin (RBV), one or more nucleos(t)ide inhibitors, including for example nucleoside inhibitor-1 (NI-1), 2′C-methyl adenosine (2′CMeA), sofosbuvir (SOF), or non-nucleoside inhibitor targeting site A or B (NNI-A or NNI-B) are provided. The methods may involve determining the genotype of the HCV or the phenotype of the HCV with respect to the inhibitor susceptibility. The methods may further include the selection of a suitable treatment based on the genotype or phenotype determined.

### CROSS REFERENCE TO RELATED APPLICATIONS

This application is a divisional application of U.S. patent application Ser. No. 14/217,410 filed Mar. 17, 2014, which claims priority to U.S. Provisional Application No. 61/802,212, filed Mar. 15, 2013; U.S. Provisional Application No. 61/831,134, filed Jun. 4, 2013; and U.S. Provisional Application No. 61/839,947, filed Jun. 27, 2013. The entire contents of each of these applications are incorporated herein by reference.

### FIELD

Embodiments of the present invention relate to methods for determining the susceptibility of a hepatitis C virus (“HCV”) or HCV population to HCV inhibitors. The methods for determining susceptibility include genotypic or phenotypic methods.

### BACKGROUND OF THE INVENTION

HCV affects an estimated 170 million people worldwide, including 4 million Americans, or approximately 1% of the United States population making it the most common blood-borne illness. HCV infection becomes a chronic condition in approximately 55-85% of patients. Late complications of chronic HCV infection include cirrhosis of the liver, hepatocellular carcinoma, and mortality. There is no effective vaccine for the prevention of HCV infection.

HCV is an enveloped virus containing a positive sense, linear, single-stranded RNA genome of approximately 9,000 nucleotides (9 kb). HCV is classified in the family Flaviviridae along with the flaviviruses and pestiviruses. The single open reading frame of the HCV genome is translated to produce a single protein product, which is then further processed to produce smaller active proteins, including three structural proteins (nucleocapsid (C) and two envelope glycoproteins (E1 and E2)) and seven non-structural proteins (including, among others, a serine protease (non-structural protein 3 (NS3)), cofactor (non-structural protein 4A (NS4A)), non-structural protein 5A (NS5A), and RNA dependent RNA polymerase (non-structural protein 5B (NSSB)).

HCV strains are grouped by “genotype” based on phylogeny (genetic sequence) into one of six genotypes (i.e., 1-6), which are further characterized into several different subtypes within a genotype (e.g., 1a, 1b, 1c). Infection with one HCV genotype does not necessarily provide immunity to the patient against HCV of that genotype or any other genotypes, and therefore, concurrent infection with more than one HCV genotype isolates is possible. In large part, HCV genotypes are geographically distinct. In North America, Europe, and Japan, HCV genotype 1 (GT1) is most prevalent. Within genotype 1 HCV, subtypes 1a and 1b are more prevalent, and subtype 1c is only a minor component. In other regions, however, HCV genotypes other than genotype 1 (non-GT1) are prevalent.

Prior to the approval of direct-acting antiviral (DAAs) agents for HCV, the standard of care for HCV infection relied on indirect suppression of viral replication through immune modulation in response to 24-48 weeks of treatment with pegylated interferon alpha (PEG-IFN) in combination with ribavirin (RBV). Response to treatment varies among patients, as discussed further below, with approximately 40-60% of patients achieving a sustained suppression of viral replication (sustained virologic response, SVR). Not all HCV-infected patients with an initial response to standard PEG-IFN/RBV therapy sustain their responses, as evidenced by rising levels of detectable HCV RNA in plasma. Due to varied efficacy and the low tolerability of PEG-IFN/RBV therapy, a large number of new antiviral agents that directly target HCV replication are being evaluated in preclinical development programs and clinical trials, and 4 DAAs have now been approved (boceprevir, telaprevir, simeprevir, sofosbuvir). Approved DAAs include a polymerase inhibitor (sofosbuvir) and protease inhibitors (boceprevir, telaprevir, simeprevir). Additional inhibitors targeting the viral protease, the non-structural protein 5A, or the RNA-dependent RNA polymerase (RdRp), encoded by the NS3, NS5A, and NS5B regions of the HCV genome, respectively, are furthest along in development (See list of drugs in development, for example, at http://www.hcvadvocate.org/hepatitis/hepC/Quick_Ref_Guide.pdf).

The NS5B region of HCV is 1,773 nucleotides in length and encodes the HCV RdRp enzyme. The HCV RdRp enzyme “copies” the HCV RNA genome and produces both positive and negative sense HCV RNA, thus RdRp is essential for viral replication. A number of nucleos(t)ide inhibitors (NIs) and small molecule non-nucleoside inhibitors (NNIs) are currently being developed, and sofosbuvir (SOF) was recently approved for use in combination therapy. NIs act by competing with the natural substrates (ribonucleoside triphosphates) of RdRp for binding at the active site. NNIs bind allosterically and inhibit RdRp activity by non-competitive mechanisms. NNIs may be further grouped into several subclasses that are distinguished based on their chemical structure and target binding sites. Resistance to specific RdRp inhibitors has been reported as being associated with certain amino acid mutations located within the enzyme that limit inhibitor binding either by altering the RdRp structure (e.g., NNIs) or by improving the ability of the RdRp to discriminate between the inhibitor and natural substrates (e.g., NIs).

Because currently available HCV inhibitors affect GT1 HCV and HCV of other genotypes (i.e., non-GT1 HCV) differently, different preferred treatment regimens have been implemented. The standard of care for GT1 virus infection had been pegylated interferon alpha (PEG-IFN) in combination with ribavirin (RBV), prior to the approval of the use of different protease inhibitors, or the nucleoside inhibitor SOF, in combination with PEG-IFN/RBV. Currently, the standard of care for GT1, GT4, GT5, and GT6 HCV is pegylated interferon alpha (PEG-IFN) in combination with ribavirin (RBV) and sofosbuvir (SOVALDI, Gilead Sciences, SOF), and the standard of care for GT2 and GT3 HCV is RBV and SOF. The standard of care for GT1 HCV in patients not eligible to receive IFN includes SOF, RBV, and the protease inhibitor simeprevir (OLYSIO, Janssen Therapeuticas, Titusville, N.J.). It had been observed previously that patients infected with non-GT1 viruses typically achieve higher sustained virologic response (SVR) rates following PEG-IFN/RBV treatment compared to those with GT1 viruses. Better SVRs among non-GT1 viruses compared to GT1 viruses also have been observed in clinical trials with nucleos(t)ide polymerase inhibitors (NIs). The reasons for differential responses between genotypes are unclear, but could include viral properties and relative inhibitor susceptibilities. In any event, it is highly desirable to have methods that determine the inhibitor susceptibility of the HCV infecting an individual in order to determine the best treatment regimen for the individual.

The inhibitor ribavirin, which is part of the current standard of care for HCV infection, is a prodrug, which when metabolized, resembles purine RNA nucleotides and interferes with RNA metabolism required for viral replication. The mechanism of how ribavirin affects viral replication is unknown. Although many mechanisms have been proposed for ribavirin, none of these has been proven to date and it may be that there are multiple mechanisms responsible for its actions. Ribavirin is not substantially incorporated into DNA, but does have a dose-dependent inhibiting effect on DNA synthesis, as well as having other effects on gene expression. It is a cause of anemia in patients as well. Significant teratogenic effects have been noted in non-primate animal species on which ribavirin has been tested, and it has been noted that ribavirin has a long half-life in the body. Ribavirin also may be toxic to cells in currently used susceptibility assays, making it difficult to accurately determine its effect on a particular HCV population. It would be advantageous to have methods that accurately and efficiently determine the susceptibility of an HCV infecting an individual to ribavirin in order to determine whether it is appropriate to include ribavirin in the treatment regimen for the individual, or potentially to determine treatment duration.

The inhibitor sofosbuvir, which is also part of the current standard of care for HCV infection, is an NS5B polymerase inhibitor. Sofosbuvir is a nucleotide prodrug that undergoes intracellular metabolism to form the pharmacologically active uridine analog triphosphate (GS-461203). Sofosubuvir mimics a nucleotide but acts as a chain terminator. When sofosbuvir is substituted for the normal nucleotide, the virus cannot replicate. Reported adverse effects of sofosbuvir when administered in combination with ribavirin were fatigue and headache, and the most common adverse effects of sofosbuvir when administered in combination with PEG-IFN were fatigue, headache, nausea, insomnia, and anemia.

Although several of the currently available inhibitors have been shown to be effective in terms of inhibiting viral replication, they are susceptible to the development of resistance of the virus due to its rapid mutation rate which results in the rapid emergence of mutant HCV having reduced susceptibility to an antiviral therapeutic upon administration of such drug to infected individuals. This reduced susceptibility to a particular drug renders treatment with that drug ineffective for the infected individual. For this reason, it is important for practitioners to be able to monitor drug susceptibility in order to determine the most appropriate treatment regimen for each HCV infected individual.

Therefore, there is a need for methods and compositions for the efficient and accurate determination of susceptibility to drugs, such as ribavirin and nucleos(t)ide inhibitors, targeting HCV polypeptides. The desired methods and compositions would facilitate the evaluation of (a) natural variation in HCV inhibitor susceptibility and/or (b) differences in pre-treatment, on-treatment, and post-treatment inhibitor susceptibility that would signify the emergence and persistence or decay of HCV inhibitor resistant populations. What is also needed are methods that can be used to evaluate the relative replication capacity (RC) of HCV populations. These and other needs are met by the present invention.

### SUMMARY OF THE INVENTION

The present application provides methods and compositions for the efficient and accurate determination of susceptibility of mixed hepatitis C virus (HCV) populations to HCV inhibitors.

Methods are provided for selecting a treatment for a patient having a hepatitis C virus (HCV) infection. In certain embodiments, the methods may include the steps of obtaining a biological sample from the patient, wherein the biological sample comprises an HCV or HCV population from the patient; determining the genotype of the HCV or HCV population; and determining the appropriate course of treatment, which could include inhibitors and/or treatment duration, based on the genotype(s) of the HCV determined. The treatment may include, in some embodiments, a ribavirin or a nucleoside inhibitor containing treatment regimen if the HCV or HCV population comprises a substantial amount of a genotype 2 (GT2) HCV, genotype 3 (GT3) HCV, genotype 4 (GT4) HCV, or a combination thereof. The treatment may include, in some embodiments, a sofosbuvir containing treatment regimen if the HCV or HCV population comprises a substantial amount of a GT2 HCV. The the treatment may not include sofosbuvir or may include a longer period of sofosbuvir treatment if the HCV or HCV population comprises a substantial amount of a GT3 HCV, GT4 HCV, or a combination thereof. The treatment may not include a non-nucleoside inhibitor targeting site B (NNI-B) or may contain a longer treatment with that inhibitor if the HCV or HCV population comprises a GT2 HCV or GT3 HCV, and the treatment may not include a non-nucleoside inhibitor targeting site A (NNI-A) or may contain a longer treatment with that inhibitor if the HCV or HCV population comprises a GT2 HCV.

Also provided are methods for determining the susceptibility of a hepatitis C virus (HCV) or HCV virus population to an HCV inhibitor, wherein the HCV inhibitor is interferon (IFN), ribavirin (RBV), one or more nucleos(t)ide inhibitors, including for example nucleos(t)ide inhibitors such as nucleoside inhibitor-1 (NI-1), 2′C-methyl adenosine (2′CMeA), sofosbuvir (SOF), or non-nucleoside inhibitor targeting site A or B (NNI-A or NNI-B). In certain embodiments, the methods may include the steps of determining the genotype of the HCV or HCV population; and determining that the HCV or HCV population is likely to have increased susceptibility to ribavirin as compared to a reference virus if the HCV or HCV population comprises a genotype 2 (GT2) HCV, genotype 3 (GT3) HCV, genotype 4 (GT4) HCV, or a combination thereof, determining that the HCV or HCV population is likely to have increased susceptibility to sofosbuvir if the HCV or HCV population comprises a GT2 HCV; determining that the HCV or HCV population is likely to have decreased susceptibility to sofosbuvir if the HCV or HCV population comprises a GT3 HCV, GT4 HCV, or a combination thereof, determining that the HCV or HCV population is likely to have decreased susceptibility to non-nucleoside inhibitor targeting site B (NNI-B) if the HCV or HCV population comprises a GT2 HCV, GT3 HCV, or a combination thereof, or determining that the HCV or HCV population is likely to have decreased susceptibility to a non-nucleoside inhibitor targeting site A (NNI-A) if the HCV or HCV population comprises a GT2 HCV.

Methods are provided for determining the susceptibility of a hepatitis C virus (HCV) or HCV population to an HCV polymerase inhibitor. In certain embodiments, the HCV inhibitor is interferon (IFN), ribavirin (RBV), one or more nucleos(t)ide inhibitors, including for example nucleos(t)ide inhibitor such as nucleoside inhibitor-1 (NI-1), 2′C-methyl adenosine (2′CMeA), sofosbuvir (SOF), or non-nucleoside inhibitor targeting site A, B, C, or D (NNI-A, NNI-B, NNI-C, NNI-D). The methods may comprise the steps of introducing into a cell a resistance test vector comprising a patient derived segment from the HCV viral population, wherein the cell or the resistance test vector comprises an indicator nucleic acid that produces a detectable signal that is dependent on the HCV; measuring the expression of the indicator gene in the cell in the absence or presence of increasing concentrations of the HCV inhibitor; developing a standard curve of drug susceptibility for the HCV inhibitor, wherein the IC₅₀ fold change value, IC₉₅ fold change value, both, or the slope are detected in the standard curve; comparing the IC₅₀ fold change value, IC₉₅ fold change value, or both of the HCV population to an IC₅₀ fold change value, IC₉₅ fold change value, or both for a control HCV population or comparing the slope of the standard curve of the HCV population to the slope of the standard curve for a control HCV population; and determining that the HCV population comprises HCV particles with an increased susceptibility to the HCV inhibitor when the IC₅₀ fold change value, IC₉₅ fold change value, or both are lower for the HCV population as compared to the IC₅₀ fold change value, IC₉₅ fold change value, or both for the control HCV population or determining that the HCV population comprises HCV particles with a reduced susceptibility to the HCV inhibitor when the slope of the standard curve of the HCV population is decreased as compared to the standard curve of the control population. The HCV inhibitor may be, for example, an interferon (IFN), ribavirin (RBV), one or more nucleos(t)ide inhibitors, including for example nucleoside inhibitor-1 (NI-1), 2′C-methyl adenosine (2′CMeA), sofosbuvir (SOF), or non-nucleoside inhibitor targeting site A or B (NNI-A or NNI-B). In certain specific embodiments, the control HCV population comprises Con1 HCV or H77 HCV. In certain other specific embodiments, the control HCV population is an HCV population from the patient before treatment with the HCV inhibitor. In certain embodiments, the resistance test vector comprises the patient derived segment and the indicator nucleic acid. In some embodiments, the patient derived segment comprises the NS5B region of the HCV. In certain embodiments, the indicator gene comprises a luciferase gene. In certain embodiments of these methods, the host cells are Huh7 cells.

In certain embodiments, the methods are used to facilitate the determination of a suitable treatment regimen for a patient. In certain embodiments, the methods further comprise determining the ratio of the IC₉₅ fold change value to the IC₅₀ fold change value is detected, wherein a change in the ratio indicates a change in the susceptibility of the HCV to the inhibitor. In certain embodiments, the methods are used to facilitate the determination of a suitable treatment regimen for a patient. These data described herein may be useful to inform clinical trial design, pre-treatment decisions (e.g., drugs to use, number of drugs to combine, treatment duration), as well as for evaluating resistance. In particular, phenotypic data, in conjunction with clinical outcome data, may further strengthen the utility of the assay e.g. for developing clinical cut offs.

### BRIEF DESCRIPTION OF THE FIGURES

Non-limiting embodiments of the methods of the invention are exemplified in the following figures.

FIG. 1 is a schematic diagram of a phenotypic assay for determining HCV inhibitor susceptibility. The diagram uses as an example that the HCV inhibitor is targeting the HCV protease NS3, nonstructural protein 5A (NS5A), or polymerase NS5B. Therefore, in this example, the NS3, NS5A, or NS5B region of the test population is included in the replicon test vector.

FIG. 2 is a graph showing a representative HCV inhibitor susceptibility curve, plotting the concentration of the HCV inhibitor on the x-axis and the fold change in susceptibility as a percent inhibition on the y-axis. The IC₅₀ and IC₉₅ are indicated. The slope may be calculated by curve fitting based on the log sigmoid function. For example, inhibition is equal to top−(top+base) divided by (1+concentration/center)̂slope). Simplified, the slope is equal to the log(95/100−95)/Δx. Δx is equal to the log(IC₉₅)−log(IC₅₀).

FIG. 3 is a table showing the accuracy, precision, reproducibility, sensitivity, and linearity of the present assay.

FIG. 4 further shows the inter-assay variation using replicons containing NS5B populations from patient samples. Representative reproducibility data are shown in the two graphs for NNI-A susceptibility in the left graph and replication capacity in the right graph. The data from assay 1 is plotted on the x axis, and data from assay 2 is plotted on the y axis. Fold changes of 2 or greater were found to be reproducibly detected with this assay.

FIGS. 5 and 6 demonstrate the variation in susceptibility to a nucleoside inhibitor (NI) and ribavirin of non-GT1 viruses as compared to GT1 viruses. The raw data is shown in FIG. 5, and the data is shown graphically in FIG. 6. In FIG. 6A, GT1 virus data is shown. NonGT-1 virus susceptibility data is shown in FIG. 6B. In both FIGS. 6A and 6B, the IC fold change is plotted on the y axis, and the inhibitor and IC value are indicated on the x axis. In FIGS. 6C and 6D, the IC50 or IC95 fold change, respectively, are plotted on the y axis, and the inhibitors and HCV genotype are plotted on the x axis. The dotted lines show the range of IC that is within 2-fold of the IC of the Con1 reference. The light asterisks indicate a significant difference from GT1, and the dark asterisks indicate a significant difference between subtypes (Wilcoxon rank sum test). The IC FC range for IFN, RBV, and NI is 5, 25, and 10, respectively. The susceptibility for IFN is 3>1, 2>4. The susceptibility for RBV is 3>4>2>1. The susceptibility for NI is 3>2>1>4.

FIG. 7 shows three tables demonstrating the variation in susceptibility of HCV viruses of different genotypes to an interferon (IFN), ribavirin (RBV), nucleoside inhibitor (NI-1), 2′C-methyl adenosine (2′CMeA), sofosbuvir (SOF). The raw data is shown in the tables in FIG. 7. The inhibitor and genotype are indicated, as well as the number of viruses of the indicated genotype (“number of values”). The median, maximum, minimum, and range of IC₅₀ fold changes (compared to the IC50 of reference virus) for each virus genotype for each inhibitor are shown. The range of IC₅₀ fold changes between all of the tested genotypes is shown at the bottom of the tables.

FIG. 8 is a table showing the significance of the variation in susceptibility to an interferon, ribavirin, nucleoside inhibitor, 2′C-methyl adenosine (2′CMeA), sofosbuvir (SOF) between the viruses of different genotypes, using a Wilcoxon rank sum test. The inhibitor is shown in the first column, and the genotypes of the two viruses that are being compared are shown in the second and third columns. The statistical significance of the difference in susceptibilities (IC₅₀ fold change) between the two viruses is shown in the fourth column, and those that have a P<0.05 are indicated with “Yes” to show that the difference was statistically significant.

FIG. 9 demonstrates the variation in susceptibility to an interferon, ribavirin, and nucleos(t)ide inhibitors such as NI-1, 2′C-methyl adenosine (2′CmeA), sofosbuvir (SOF) of viruses of different genotypes GT1 (a/b), GT2 (a/b/k), GT3 (a), and GT4 (a/d/n/unknown). The IC₅₀ fold change as compared to a reference virus value is plotted on the y axis, and the inhibitor and genotype of the virus tested are indicated on the x axis. The genotypes indicated by a number without a subtype on the x axis indicate that the results shown in that column include the results for all of the subtypes evaluated from that genotype (e.g., compare the dots shown in the 1 column with the dots shown in 1a column and 1b column).

FIG. 10 demonstrates the variation in susceptibility to non-nucleoside inhibitors of viruses of different genotypes GT1, GT2, GT3, and GT4. In FIGS. 10A and 10B, the IC₅₀ or IC₉₅ fold change to the Con1 reference virus, respectively, are plotted on the y axis, and the inhibitors and HCV genotype are plotted on the x axis. The dotted lines show the range of IC that is within 2-fold of the IC of the Con1 reference. The light asterisks indicate a significant difference from GT1, and the dark asterisks indicate a significant difference between subtypes (Wilcoxon rank sum test). The IC FC range for NNI-A, NNI-B, and NNI-D is 827, >8.2, and 152, respectively. The susceptibility for NNI-A is 1, 3, 4>2. The susceptibility for NNI-B is (1)>4>2, 3. The susceptibility for NNI-D is 1, 3>2, 4.

### DETAILED DESCRIPTION OF THE INVENTION

The present invention provides, inter alia, methods for determining the susceptibility of an HCV population to an anti-HCV drug or for determining replication capacity of an HCV infecting a patient. The methods, and compositions useful in performing the methods, are described further below.

### Definitions and Abbreviations

The following terms are herein defined as they are used in this application:

“PCR” is an abbreviation for “polymerase chain reaction.”

“HCV” is an abbreviation for hepatitis C virus. In certain embodiments, HCV refers to HCV genotype 1. In certain embodiments, HCV refers to HCV genotype 1a, 1b, 2, 2a, 2b, 3, or 4.

The amino acid notations used herein for the twenty genetically encoded L-amino acids are conventional and are as follows:

TABLE 1

| One Letter Abbreviation | Three Letter Abbreviation | Amino Acid |
|---|---|---|
| A | Ala | Alanine |
| N | Asn | Asparagine |
| R | Arg | Arginine |
| D | Asp | Aspartic acid |
| C | Cys | Cysteine |
| Q | Gln | Glutamine |
| E | Glu | Glutamic acid |
| G | Gly | Glycine |
| H | His | Histidine |
| I | Ile | Isoleucine |
| L | Leu | Leucine |
| K | Lys | Lysine |
| M | Met | Methionine |
| F | Phe | Phenylalanine |
| P | Pro | Proline |
| S | Ser | Serine |
| T | Thr | Threonine |
| W | Trp | Tryptophan |
| Y | Tyr | Tyrosine |
| V | Val | Valine |

Unless noted otherwise, when polypeptide sequences are presented as a series of one-letter and/or three-letter abbreviations, the sequences are presented in the N→C direction, in accordance with common practice. Individual amino acids in a sequence are represented herein as AN, wherein A is the standard one letter symbol for the amino acid in the sequence, and N is the position in the sequence. Mutations are represented herein as A₁NA₂, wherein A₁ is the standard one letter symbol for the amino acid in the reference protein sequence, A₂ is the standard one letter symbol for the amino acid in the mutated protein sequence, and N is the position in the amino acid sequence. For example, a G25M mutation represents a change from glycine to methionine at amino acid position 25. Mutations may also be represented herein as N A₂, wherein N is the position in the amino acid sequence and A₂ is the standard one letter symbol for the amino acid in the mutated protein sequence (e.g., 25M, for a change from the wild-type amino acid to methionine at amino acid position 25). Additionally, mutations may also be represented herein as A₁NX, wherein A₁ is the standard one letter symbol for the amino acid in the reference protein sequence, N is the position in the amino acid sequence, and X indicates that the mutated amino acid can be any amino acid (e.g., G25X represents a change from glycine to any amino acid at amino acid position 25). This notation is typically used when the amino acid in the mutated protein sequence is not known, if the amino acid in the mutated protein sequence could be any amino acid, except that found in the reference protein sequence, or if the amino acid in the mutated position is observed as a mixture of two or more amino acids at that position. The amino acid positions are numbered based on the full-length sequence of the protein from which the region encompassing the mutation is derived. Representations of nucleotides and point mutations in DNA sequences are analogous. In addition, mutations may also be represented herein as A₁NA₂A₃A₄, for example, wherein A₁ is the standard one letter symbol for the amino acid in the reference protein sequence, N is the position in the amino acid sequence, and A₂, A₃, and A₄ are the standard one letter symbols for the amino acids that may be present in the mutated protein sequences.

The abbreviations used throughout the specification to refer to nucleic acids comprising specific nucleobase sequences are the conventional one-letter abbreviations. Thus, when included in a nucleic acid, the naturally occurring encoding nucleobases are abbreviated as follows: adenine (A), guanine (G), cytosine (C), thymine (T) and uracil (U). Unless specified otherwise, single-stranded nucleic acid sequences that are represented as a series of one-letter abbreviations, and the top strand of double-stranded sequences, are presented in the 5′→3′ direction.

As used herein, the phrase “phenotypic assay” is a test that measures a phenotype of a particular virus, such as, for example, HCV, or a population of viruses, such as, for example, the population of HCV infecting a subject. The phenotypes that can be measured include, but are not limited to, the resistance or susceptibility of a virus, or of a population of viruses, to a specific chemical or biological anti-viral agent or that measures the replication capacity of a virus.

As used herein, a “genotypic assay” is an assay that determines a genotype of an organism or population of organisms (e.g., genotype 1, 2, 3, 4), a genotype subtype of an organism or a population of organisms (e.g., genotype 1a, 1b, 2a, 2b). In certain embodiments, the genotypic assay involves determination of the nucleic acid sequence of certain gene or genes, or relevant sequences that reflect a particular genotype or genotype subtype.

As used herein, the term “mutation” refers to a change in an amino acid sequence or in a corresponding nucleic acid sequence relative to a reference nucleic acid or polypeptide. For certain embodiments of the invention, the reference nucleic acid is that of a Con1 HCV for comparison with an HCV genotype 1b population or H77 HCV for comparison with an HCV genotype 1a population. Likewise, the reference polypeptide is that encoded by the Con1 or H77 HCV nucleic acid sequence. Alternatively, the reference nucleic acid or polypeptide may be from a patient population before treatment with an HCV inhibitor. Although the amino acid sequence of a peptide can be determined directly by, for example, Edman degradation or mass spectroscopy, more typically, the amino sequence of a peptide is inferred from the nucleotide sequence of a nucleic acid that encodes the peptide. Any method for determining the sequence of a nucleic acid known in the art can be used, for example, Maxam-Gilbert sequencing (Maxam et al., 1980, Methods in Enzymology 65:499), dideoxy sequencing (Sanger et al., 1977, Proc. Natl. Acad. Sci. USA 74:5463) or hybridization-based approaches (see e.g., Sambrook et al, 2001, Molecular Cloning: A Laboratory Manual, Cold Spring Harbor Laboratory, 3.sup.rd ed., NY; and Ausubel et al, 1989, Current Protocols in Molecular Biology, Greene Publishing Associates and Wiley Interscience, NY). As used herein, the terms “position” and “codon” are used interchangeably to refer to a particular amino acid in the sequence. In certain embodiments, a mutation is known to be associated with changes in drug susceptibility. For example, certain NS5B mutations are associated with reductions in susceptibility to nucleos(t)ide inhibitors (NI; e.g., S282T mutants) or non-nucleoside polymerase inhibitors targeting site A (NNI-A; e.g., L392I and P495A/L mutants), site B (NNI-B; e.g., M423T), site C (NNI-C; e.g., C316Y and Y448H), or site D (NNI-D; e.g., C316Y).

As used herein, the term “mutant” refers to a virus, gene, or protein having a sequence that has one or more changes relative to a reference virus, gene, or protein. The terms “peptide,” “polypeptide,” and “protein” are used interchangeably throughout. Similarly, the terms “polynucleotide,” “oligonucleotide,” and “nucleic acid” are used interchangeably throughout.

The term “wild-type” is used herein to refer to a viral genotype that does not comprise a mutation known to be associated with changes in drug susceptibility (reductions or increases). As used herein, the terms “drug susceptibility” and “inhibitor susceptibility” are used interchangeably.

As used herein, the term “susceptibility” refers to a virus's response to a particular drug. A virus that has decreased or reduced susceptibility to a drug may be resistant to the drug or may be less vulnerable to treatment with the drug. By contrast, a virus that has increased or enhanced susceptibility (hyper-susceptibility) to a drug is more vulnerable to treatment with the drug. In certain embodiments, the methods disclosed for determining susceptibility may be used by a medical provider to facilitate the determination of a proper treatment regimen for a patient.

As used herein, the phrase “a substantial amount” of an HCV of a given genotype in a sample refers to an amount of HCV within the sample such that treatment of the sample with an inhibitor that is effective for the treatment of that HCV genotype is also effective to reduce the amount of viable HCV in the sample.

The term “IC₉₅” refers to the concentration of drug in the sample needed to suppress the reproduction of the disease causing microorganism (e.g., HCV) by 95%. The term “IC₅₀” refers to the concentration of drug in the sample needed to suppress the reproduction of the disease causing microorganism by 50%.

As used herein, the term “fold change” is a numeric comparison of the drug susceptibility of a patient virus and a reference virus. For example, the ratio of a mutant HCV IC₅₀ to the drug-sensitive reference HCV IC₅₀ is a fold change. A fold change of 1.0 indicates that the patient virus exhibits the same degree of drug susceptibility as the drug-sensitive reference virus. A fold change less than 1 indicates the patient virus is more sensitive than the drug-sensitive reference virus. A fold change greater than 1 indicates the patient virus is less susceptible than the drug-sensitive reference virus. A fold change equal to or greater than the clinical cutoff value means the patient virus has a lower probability of response to that drug. A fold change less than the clinical cutoff value means the patient virus is sensitive to that drug.

The phrase “clinical cutoff value” refers to a specific point at which drug sensitivity ends. It is defined by the drug susceptibility level at which a patient's probability of treatment failure with a particular drug significantly increases. The cutoff value is different for different anti-viral agents, as determined in clinical studies. Clinical cutoff values are determined in clinical trials by evaluating resistance and outcomes data. Phenotypic drug susceptibility is measured at treatment initiation. Treatment response, such as change in viral load, is monitored at predetermined time points through the course of the treatment. The drug susceptibility is correlated with treatment response, and the clinical cutoff value is determined by susceptibility levels associated with treatment failure (statistical analysis of overall trial results).

A virus may have an “increased likelihood of having reduced susceptibility” to an anti-viral treatment if the virus has a property, for example, a genotype or a mutation, that is correlated with a reduced susceptibility to the anti-viral treatment. A property of a virus is correlated with a reduced susceptibility if a population of viruses having the property is, on average, less susceptible to the anti-viral treatment than an otherwise similar population of viruses lacking the property. Thus, the correlation between the presence of the property and reduced susceptibility need not be absolute, nor is there a requirement that the property is necessary (i.e., that the property plays a causal role in reducing susceptibility) or sufficient (i.e., that the presence of the property alone is sufficient) for conferring reduced susceptibility.

The term “% sequence homology” is used interchangeably herein with the terms “% homology,” “% sequence identity,” and “% identity” and refers to the level of amino acid sequence identity between two or more peptide sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 60, 70, 80, 85, 90, 95, 98%, or more sequence identity to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at http://www.ncbi.nlm.nih.gov/BLAST/. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See Altschul, et al, 1997.

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded apolar amino acids include Ala (A), Gly (G), Ile (I), Leu (L), Met (M) and Val (V).

“Hydrophilic amino acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Arg (R), Asn (N), Asp (D), Glu (E), Gln (Q), His (H), Lys (K), Ser (S) and Thr (T).

“Hydrophobic amino acid” refers to an amino acid exhibiting a hydrophobicity of greater than zero according to the normalized consensus hydrophobicity scale of Eisenberg et al., 1984, J. Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic amino acid” refers to a hydrophilic amino acid having a side chain pK value of greater than 7. Basic amino acids typically have positively charged side chains at physiological pH due to association with hydronium ion. Genetically encoded basic amino acids include Arg (R), His (H) and Lys (K).

The term “resistance test vector,” as used herein, refers to one or more nucleic acids comprising a patient-derived segment and an indicator gene. In the case where the resistance test vector comprises more than one nucleic acid, the patient-derived segment may be contained in one nucleic acid and the indicator gene in a different nucleic acid. For example, the indicator gene and the patient-derived segment may be in a single vector, or may be in separate vectors. The DNA or RNA of a resistance test vector may thus be contained in one or more DNA or RNA molecules and may be introduced as one or more DNA or RNA molecules into a host cell. The term “patient-derived segment,” as used herein, refers to one or more nucleic acids that comprise an HCV nucleic acid sequence corresponding to a nucleic acid sequence of an HCV infecting a patient, where the nucleic acid sequence encodes an HCV gene product that is the target of an anti-HCV drug. A “patient-derived segment” can be prepared by an appropriate technique known to one of skill in the art, including, for example, molecular cloning or polymerase chain reaction (PCR) amplification from viral DNA or complementary DNA (cDNA) prepared from viral RNA, present in the cells (e.g., peripheral blood mononuclear cells, PBMC), serum, or other bodily fluids of infected patients. A “patient-derived segment” is preferably isolated using a technique where the HCV infecting the patient is not passed through culture subsequent to isolation from the patient, or if the virus is cultured, then by a minimum number of passages to reduce or essentially eliminate the selection of mutations in culture. The term “indicator,” “indicator nucleic acid,” or “indicator gene,” as used herein, refers to a nucleic acid encoding a protein, DNA structure, or RNA structure that either directly or through a reaction gives rise to a measurable or noticeable aspect, e.g., a color or light of a measurable wavelength or, in the case of DNA or RNA used as an indicator, a change or generation of a specific DNA or RNA structure. A preferred indicator gene is luciferase. Other indicator genes are described below and are well known in the art.

### Genotypic Methods of Determining Susceptibility to HCV Inhibitors

In certain embodiments, the methods may include the steps of obtaining a biological sample from the patient, wherein the biological sample comprises an HCV or HCV population from the patient; determining the genotype of the HCV or HCV population; and determining the appropriate course of treatment based on the genotype(s) of the HCV determined. The treatment may include ribavirin if the HCV or HCV population comprises a substantial amount of a genotype 2 (GT2) HCV, genotype 3 (GT3) HCV, genotype 4 (GT4) HCV, or a combination thereof. The treatment may include sofosbuvir if the HCV or HCV population comprises a substantial amount of a GT2 HCV, and the treatment may not include sofosbuvir if the HCV or HCV population comprises a substantial amount of a GT3 HCV, GT4 HCV, or a combination thereof.

Also provided are methods for determining the susceptibility of a hepatitis C virus (HCV) or HCV virus population to an HCV inhibitor, wherein the HCV inhibitor is an interferon (IFN), ribavirin (RBV), one or more nucleos(t)ide inhibitors, including for example nucleoside inhibitor-1 (NI-1), 2′C -methyl adenosine (2′CMeA), or sofosbuvir (SOF). In certain embodiments, the methods may include the steps of determining the genotype of the HCV or HCV population; and determining that the HCV or HCV population is likely to have increased susceptibility to ribavirin as compared to a reference virus if the HCV or HCV population comprises a genotype 2 (GT2) HCV, genotype 3 (GT3) HCV, genotype 4 (GT4) HCV, or a combination thereof, determining that the HCV or HCV population is likely to have increased susceptibility to sofosbuvir if the HCV or HCV population comprises a GT2 HCV; or that the HCV or HCV population is likely to have decreased susceptibility to sofosbuvir if the HCV or HCV population comprises a GT3 HCV, GT4 HCV, or a combination thereof.

The genotype of an HCV or HCV population may be determined by any method known by those of ordinary skill in the art (e.g., sequencing). Any method for determining the sequence of a nucleic acid known in the art can be used, for example, Maxam-Gilbert sequencing (Maxam et al., 1980, Methods in Enzymology 65:499), dideoxy sequencing (Sanger et al., 1977, Proc. Natl. Acad. Sci. USA 74:5463) or hybridization-based approaches (see e.g., Sambrook et al., 2001, Molecular Cloning: A Laboratory Manual, Cold Spring Harbor Laboratory, 3. sup. rd ed., NY; and Ausubel et al., 1989, Current Protocols in Molecular Biology, Greene Publishing Associates and Wiley Interscience, NY).

The determination of specific nucleic acid sequences can be accomplished by a variety of methods including, but not limited to, restriction-fragment-length-polymorphism detection based on allele-specific restriction-endonuclease cleavage (Kan and Dozy, 1978, Lancet ii:910-912), mismatch-repair detection (Faham and Cox, 1995, Genome Res 5:474-482), binding of MutS protein (Wagner et al., 1995, Nucl Acids Res 23:3944-3948), denaturing-gradient gel electrophoresis (Fisher et al., 1983, Proc. Natl. Acad. Sci. 80:1579-83), single-strand-conformation-polymorphism detection (Orita et al., 1983, Genomics 5:874-879), RNAase cleavage at mismatched base-pairs (Myers et al., 1985, Science 230:1242), chemical (Cotton et al., 1988, Proc. Natl. Acad. Sci. U.S.A. 85:4397-4401) or enzymatic (Youil et al., 1995, Proc. Natl. Acad. Sci. U.S.A. 92:87-91) cleavage of heteroduplex DNA, methods based on oligonucleotide-specific primer extension (Syvanen et al., 1990, Genomics 8:684-692), genetic bit analysis (Nikiforov et al., 1994, Nucl Acids Res 22:4167-4175), oligonucleotide-ligation assay (Landegren et al., 1988, Science 241:1077), oligonucleotide-specific ligation chain reaction (“LCR”) (Barrany, 1991, Proc. Natl. Acad. Sci. U.S.A. 88:189-193), gap-LCR (Abravaya et al., 1995, Nucl Acids Res 23:675-682), radioactive or fluorescent DNA sequencing using standard procedures well known in the art, and peptide nucleic acid (PNA) assays (Orum et al., 1993, Nucl. Acids Res. 21:5332-5356; Thiede et al., 1996, Nucl. Acids Res. 24:983-984).

In addition, viral DNA or RNA may be used in hybridization or amplification assays to detect abnormalities involving gene structure, including point mutations, insertions, deletions and genomic rearrangements. Such assays may include, but are not limited to, Southern analyses (Southern, 1975, J. Mol. Biol. 98:503-517), single stranded conformational polymorphism analyses (SSCP) (Orita et al., 1989, Proc. Natl. Acad. Sci. USA 86:2766-2770), and PCR analyses (U.S. Pat. Nos. 4,683,202; 4,683,195; 4,800,159; and 4,965,188; PCR Strategies, 1995 Innis et al. (eds.), Academic Press, Inc.).

Such diagnostic methods can involve for example, contacting and incubating the viral nucleic acids with one or more labeled nucleic acid reagents including recombinant DNA molecules, cloned genes or degenerate variants thereof under conditions favorable for the specific annealing of these reagents to their complementary sequences. Preferably, the lengths of these nucleic acid reagents are at least 15 to 30 nucleotides. After incubation, all non-annealed nucleic acids are removed from the nucleic acid molecule hybrid. The presence of nucleic acids which have hybridized, if any such molecules exist, is then detected. Using such a detection scheme, the nucleic acid from the virus can be immobilized, for example, to a solid support such as a membrane, or a plastic surface such as that on a microliter plate or polystyrene beads. In this case, after incubation, non-annealed, labeled nucleic acid reagents of the type described above are easily removed. Detection of the remaining, annealed, labeled nucleic acid reagents is accomplished using standard techniques well-known to those in the art. The coding region sequences to which the nucleic acid reagents have annealed can be compared to the annealing pattern expected from a known coding region sequence in order to determine whether a particular genotype or sequence is present.

These techniques can easily be adapted to provide high-throughput methods for determining the length of envelope protein variable regions and/or number of envelope protein glycosylation sites in viral genomes. For example, a gene array from Affymetrix (Affymetrix, Inc., Sunnyvale, Calif.) can be used to rapidly identify genotypes of a large number of individual viruses. Affymetrix gene arrays, and methods of making and using such arrays, are described in, for example, U.S. Pat. Nos. 6,551,784, 6,548,257, 6,505,125, 6,489,114, 6,451,536, 6,410,229, 6,391,550, 6,379,895, 6,355,432, 6,342,355, 6,333,155, 6,308,170, 6,291,183, 6,287,850, 6,261,776, 6,225,625, 6,197,506, 6,168,948, 6,156,501, 6,141,096, 6,040,138, 6,022,963, 5,919,523, 5,837,832, 5,744,305, 5,834,758, and 5,631,734, each of which is hereby incorporated by reference in its entirety.

In addition, Ausubel et al., eds., Current Protocols in Molecular Biology, 2002, Vol. 4, Unit 25B, Ch. 22, which is hereby incorporated by reference in its entirety, provides further guidance on construction and use of a gene array for determining the genotypes of a large number of viral isolates. Finally, U.S. Pat. Nos. 6,670,124; 6,617,112; 6,309,823; 6,284,465; and 5,723,320, each of which is incorporated by reference in its entirety, describe related array technologies that can readily be adapted for rapid identification of a large number of viral genotypes by one of skill in the art.

Alternative diagnostic methods for the detection of gene specific nucleic acid molecules may involve their amplification, e.g., by PCR (U.S. Pat. Nos. 4,683,202; 4,683,195; 4,800,159; and 4,965,188; PCR Strategies, 1995 Innis et al. (eds.), Academic Press, Inc.), followed by the detection of the amplified molecules using techniques well known to those of skill in the art. The resulting amplified sequences can be compared to those which would be expected if the nucleic acid being amplified contained only normal copies of the respective gene in order to determine whether a gene mutation exists.

Antibodies directed against the viral gene products, i.e., viral proteins or viral peptide fragments can also be used to detect mutations in the viral proteins. Alternatively, the viral protein or peptide fragments of interest can be sequenced by any sequencing method known in the art in order to yield the amino acid sequence of the protein of interest. An example of such a method is the Edman degradation method which can be used to sequence small proteins or polypeptides. Larger proteins can be initially cleaved by chemical or enzymatic reagents known in the art, for example, cyanogen bromide, hydroxylamine, trypsin or chymotrypsin, and then sequenced by the Edman degradation method.

### Phenotypic Methods of Determining Susceptibility to HCV Inhibitors

Methods are provided for determining the susceptibility of a hepatitis C virus (HCV) population to an HCV inhibitor, comprising the steps of introducing into a cell a resistance test vector comprising a patient derived segment from the HCV viral population, wherein the cell or the resistance test vector comprises an indicator nucleic acid that produces a detectable signal that is dependent on the HCV; measuring the expression of the indicator gene in the cell in the absence or presence of increasing concentrations of the HCV inhibitor; developing a standard curve of drug susceptibility for the HCV inhibitor, wherein the IC₉₅ fold change value is detected in the standard curve; comparing the IC₉₅ fold change value of the HCV population to an IC₉₅ fold change value for a control HCV population; and determining that the HCV population comprises HCV particles with a reduced susceptibility to the HCV inhibitor when the IC₉₅ fold change is greater for the HCV population as compared to the IC₉₅ fold change for the control HCV population.

In certain aspects, the HCV inhibitor targets the HCV polymerase. The HCV inhibitor may be, for example, a nucleos(t)ide inhibitor (NI) or a non-nucleoside inhibitor (NNI). In some embodiments, the HCV is a non-nucleoside inhibitor that targets site A, B, C, or D of polymerase (NNI-A, NNI-B, NNI-C, or NNI-D). In certain aspects, the HCV inhibitor targets NS5A. In some embodiments, the HCV population and the control HCV population comprise HCV genotype 1, genotype 2, genotype 3, or genotype 4. The HCV population and the control HCV population may comprise, in certain embodiments, HCV genotype 1a, 1b, 2a, or 2b. In certain specific embodiments, the control HCV population comprises Con1 HCV or H77 HCV. In certain other specific embodiments, the control HCV population is a HCV population from the patient before treatment with the HCV inhibitor. In certain embodiments, the resistance test vector comprises the patient derived segment and the indicator nucleic acid. In some embodiments, the patient derived segment comprises the NS5B region of the HCV. In certain embodiments, the indicator gene comprises a luciferase gene. In certain embodiments of these methods, the host cells are Huh7 cells. In certain embodiments, the methods are used to facilitate the determination of a suitable treatment regimen for a patient. In certain embodiments, the methods further comprise determining the IC₅₀ fold change value, and determining the ratio of the IC₉₅ fold change value to the IC₅₀ fold change value is detected, wherein a change in the ratio indicates a change in the susceptibility of the HCV to the inhibitor.

Also provided are methods for determining the susceptibility of a hepatitis C virus (HCV) population to an HCV inhibitor, comprising the steps of introducing into a cell a resistance test vector comprising a patient derived segment from the HCV viral population, wherein the cell or the resistance test vector comprises an indicator nucleic acid that produces a detectable signal that is dependent on the HCV; measuring the expression of the indicator gene in the cell in the absence or presence of increasing concentrations of the HCV inhibitor; determining a standard curve of drug susceptibility of the HCV population to the HCV inhibitor; comparing the slope of the standard curve of the HCV population to the slope of a standard curve for a control HCV population; and determining that the HCV population comprises HCV particles with a reduced susceptibility to the HCV inhibitor when the slope of the standard curve of the HCV population is decreased as compared to the standard curve of the control population. In certain aspects, the HCV inhibitor targets the HCV polymerase. The HCV inhibitor may be, for example, a nucleos(t)ide inhibitor (NI) or a non-nucleoside inhibitor (NNI). In some embodiments, the HCV is a non-nucleoside inhibitor that targets site A, B, C, or D of the HCV polymerase (NNI-A, NNI-B, NNI-C, or NNI-D). In certain aspects, the HCV inhibitor targets NS5A. In some embodiments, the HCV population and the control HCV population comprise HCV genotype 1, genotype 2, genotype 3, or genotype 4. The HCV population and the control HCV population may comprise, in certain embodiments, HCV genotype 1a, 1b, 2a, or 2b. In certain specific embodiments, the control HCV population comprises Con1 HCV or H77 HCV. In certain other specific embodiments, the control HCV population is a HCV population from the patient before treatment with the HCV inhibitor. In certain embodiments, the resistance test vector comprises the patient derived segment and the indicator gene. In some embodiments, the patient derived segment comprises the NS5B region of the HCV. In certain embodiments, the indicator gene comprises a luciferase gene. In certain embodiments of these methods, the host cells are Huh7 cells. In certain embodiments, the methods are used to facilitate the determination of a suitable treatment regimen for a patient.

Also provided are methods for determining the susceptibility of a hepatitis C virus (HCV) population to an HCV inhibitor, comprising the steps of introducing into a cell a resistance test vector comprising a patient derived segment from the HCV viral population, wherein the cell or the resistance test vector comprises an indicator nucleic acid that produces a detectable signal that is dependent on the HCV; measuring the expression of the indicator gene in the cell in the absence or presence of increasing concentrations of the HCV inhibitor; determining a standard curve of drug susceptibility of the HCV population to the HCV inhibitor; comparing the maximum percentage inhibition of the HCV population to the maximum percentage inhibition for a control HCV population; and determining the HCV population comprises HCV particles with a reduced susceptibility to the HCV inhibitor when the maximum percentage inhibition of the HCV population is decreased as compared to the maximum percentage inhibition of the control population. In certain aspects, the HCV inhibitor targets the HCV polymerase. The HCV inhibitor may be, for example, a nucleos(t)ide inhibitor (NI) or a non-nucleoside inhibitor (NNI). In some embodiments, the HCV is a non-nucleoside inhibitor that targets site A, B, C, or D of the HCV polymerase (NNI-A, NNI-B, NNI-C, or NNI-D). In certain aspects, the HCV inhibitor targets NS5A. In certain aspects, the HCV inhibitor targets NS3. In some embodiments, the HCV population and the control HCV population comprise HCV genotype 1, genotype 2, genotype 3, or genotype 4. The HCV population and the control HCV population may comprise, in certain embodiments, HCV genotype 1a, 1b, 2a, or 2b. In certain specific embodiments, the control HCV population comprises Con1 HCV or H77 HCV. In certain other specific embodiments, the control HCV population is a HCV population from the patient before treatment with the HCV inhibitor. In certain embodiments, the resistance test vector comprises the patient derived segment and the indicator gene. In some embodiments, the patient derived segment comprises the NS5B region of the HCV. In certain embodiments, the indicator gene comprises a luciferase gene. In certain embodiments of these methods, the host cells are Huh7 cells. In certain embodiments, the methods are used to facilitate the determination of a suitable treatment regimen for a patient.

### Phenotypic Susceptibility Analysis

In certain embodiments, methods for determining HCV inhibitor susceptibility of a particular virus involve culturing a host cell comprising a patient-derived segment and an indicator gene in the presence of the HCV inhibitor, measuring the activity of the indicator gene in the host cell; and comparing the activity of the indicator gene as measured with a reference activity of the indicator gene, wherein the difference between the measured activity of the indicator gene relative to the reference activity correlates with the susceptibility of the HCV to the HCV inhibitor, thereby determining the susceptibility of the HCV to the HCV inhibitor. In certain embodiments, the activity of the indicator gene depends on the activity of a polypeptide encoded by the patient-derived segment. In preferred embodiments, the patient-derived segment comprises a nucleic acid sequence that encodes NS5B. In other embodiments, the patient-derived segment encodes the HCV protease NS3 or the NS5A protein. In certain embodiments, the patient-derived segment is obtained from the HCV.

In certain embodiments, the reference activity of the indicator gene is determined by determining the activity of the indicator gene in the absence of the HCV inhibitor. In certain embodiments, the reference activity of the indicator gene is determined by determining the susceptibility of a reference HCV to an NI or NNI. In certain embodiments, the reference activity is determined by performing a method of the invention with a standard laboratory viral segment. In certain embodiments, the standard laboratory viral segment comprises a nucleic acid sequence from HCV strain Con1 or H77.

In certain embodiments, the HCV is determined to have reduced susceptibility to the HCV inhibitor. In certain embodiments, the HCV is determined to have increased susceptibility to the HCV inhibitor. In certain embodiments, the patient-derived segment has been prepared in a reverse transcription and a polymerase chain reaction (PCR) reaction or a PCR reaction alone.

In certain embodiments, the method additionally comprises the step of infecting the host cell with a viral particle comprising the patient-derived segment and the indicator gene prior to culturing the host cell.

In certain embodiments, the indicator gene is a luciferase gene. In certain embodiments, the indicator gene is a lacZ gene. In certain embodiments, the host cell is a human cell. In certain embodiments, the host cell is a human hepatocarcinoma cell. In certain embodiments, the host cell is a Huh7 cell. In other embodiments, the host cell is a Huh7 derivative (e.g., Huh7.5, Huh7.5.1). Huh7.5 cells—human hepatocyte cell line was generated by curing a stably selected HCV replicon-containing cell line with IFN. (Blight K J, et al. J Virol 76: 13001-13014, 2002). In certain other embodiments, the host cell is a HepG2 cell, a Hep3B cell, or a derivative thereof. In certain embodiments, the host cell is derived from a human hepatoma cell line. In certain embodiments, the host cell is a primary hepatocyte (e.g., from fetal, adult, or regenerating liver). In yet other embodiments, the host cell is a lymphocyte cell (e.g., B cell, B cell lymphoma).

In another aspect, the invention provides a vector comprising a patient-derived segment and an indicator gene. In certain embodiments, the patient-derived segment comprises a nucleic acid sequence that encodes HCV NS3, NS5A, or NS5B. In certain preferred embodiments, the patient-derived segment comprises a nucleic acid sequence that encodes HCV NS5B. In certain embodiments, the activity of the indicator gene depends on the activity of the HCV NS5B.

In certain embodiments, the indicator gene is a functional indicator gene. In certain embodiments, indicator gene is a non-functional indicator gene. In certain embodiments, the indicator gene is a luciferase gene.

In another aspect, the invention provides a packaging host cell that comprises a vector of the invention. In certain embodiments, the packaging host cell is a mammalian host cell. In certain embodiments, the packaging host cell is a human host cell. In certain embodiments, the host cell is a Huh7 cell. In other embodiments, the host cell is a Huh7 derivative (e.g., Huh7.5, Huh7.5.1). Huh7.5 cells—human hepatocyte cell line was generated by curing a stably selected HCV replicon-containing cell line with IFN. (Blight K J, et al. J Virol 76: 13001-13014, 2002). In certain other embodiments, the host cell is a HepG2 cell, a Hep3B cell, or a derivative thereof. In certain embodiments, the host cell is derived from a human hepatoma cell line. In certain embodiments, the host cell is a primary hepatocyte (e.g., from fetal, adult, or regenerating liver). In yet other embodiments, the host cell is a lymphocyte cell (e.g., B cell, B cell lymphoma).

In another aspect, the invention provides a method for determining whether an HCV infecting a patient is susceptible or resistant to an HCV inhibitor. In certain embodiments, the method comprises determining the susceptibility of the HCV to the HCV inhibitor according to a method of the invention, and comparing the determined susceptibility of the HCV to HCV inhibitor with a standard curve of susceptibility of the HCV to the HCV inhibitor. In certain embodiments, a decrease in the susceptibility of the HCV to the HCV inhibitor relative to the standard curve indicates that the HCV is resistant to the HCV inhibitor. In certain embodiments, the amount of the decrease in susceptibility of the HCV to the HCV inhibitor indicates the degree to which the HCV is less susceptible to the HCV inhibitor. In certain embodiments, the HCV inhibitor is a nucleos(t)ide inhibitor (NI) or protease inhibitor. In certain embodiments, the HCV inhibitor is an interferon. In some embodiments, the interferon may comprise, for example, pegylated interferon alpha 2a, pegylated interferon alpha 2b, pegylated interferon lambda, parentals or derivatives of the above, or any member of the interferon family or derivative thereof with activity against HCV. In other embodiments, the HCV inhibitor is a non-nucleoside inhibitor (NNI) that targets site A, B, C, or D of polymerase (NNI-A, NNI-B, NNI-C, or NNI-D). In certain other aspects, the HCV inhibitor targets NS5A. In certain other aspects, the HCV inhibitor targets NS3. The HCV inhibitor may be, in some embodiments, one of the following or a combination of one or more of the following:

NS3.

BILN-2061, VX-950, SCH-503,034, SCH-900,518, TMC-435,350, R-7227 (ITMN-191), MK-5172, MK-7009, BI-201,335, BMS-650,032, BMS-824,393, PHX-1766, ACH-1625, ACH-2684, VX-985, BMS-791,325, IDX-320, GS-9256, GS-9451, ABT-450, VX-500, BIT-225

NS5A:

BMS-790,052, GSK-2336805, PPI-461, ABT-267, GS-5885, ACH-2928, AZD-7295

NS5B:

NM-283, RG-7128, R-1626, PSI-7851, IDX-184, MK-0608, PSI-7977, PSI-938, GS-6620, TMC-649,128, INX-189, VX-759, VCH-916, VX-222, ANA-598, HCV-796, GS-9190, GS-9669, ABT-333, PF-4878691, IDX-375, ABT-837,093, GSK-625,443, ABT-072.

In another aspect, the invention provides a method for determining the progression or development of resistance of an HCV infecting a patient to the HCV inhibitor. In certain embodiments, the method comprises determining the susceptibility of the HCV to the HCV inhibitor at a first time according to a method of the invention; assessing the effectiveness of the HCV inhibitor according to a method of the invention at a later second time; and comparing the effectiveness of the HCV inhibitor assessed at the first and second time. In certain embodiments, a patient-derived segment is obtained from the patient at about the first time. In certain embodiments, a decrease in the susceptibility of the HCV to the HCV inhibitor at the later second time as compared to the first time indicates development or progression of HCV inhibitor resistance in the HCV infecting the patient.

In another aspect, the present invention provides a method for determining the susceptibility of an HCV infecting a patient to the HCV inhibitor. In certain embodiments, the method comprises culturing a host cell comprising a patient-derived segment obtained from the HCV and an indicator gene in the presence of varying concentrations of the HCV inhibitor, measuring the activity of the indicator gene in the host cell for the varying concentrations of the HCV inhibitor; and determining the IC₅₀, IC₉₅, or ratio thereof of the HCV to the HCV inhibitor, wherein the IC₅₀, IC₉₅, or ratio thereof of the HCV to the HCV inhibitor indicates the susceptibility of the HCV to the HCV inhibitor. In certain embodiments, the activity of the indicator gene depends on the activity of a polypeptide encoded by the patient-derived segment. In certain embodiments, the patient-derived segment comprises a nucleic acid sequence that encodes NS5B, NS5A, and/or NS3. In certain embodiments, the IC₅₀, IC₉₅, or ratio thereof of the HCV can be determined by plotting the activity of the indicator gene observed versus the log of anti-HCV drug concentration. Alternatively, the susceptibility of the HCV to the HCV inhibitor is determined by comparing the slope or maximum inhibition of the HCV identified in the curve to the curve of a reference virus.

In still another aspect, the invention provides a method for determining the susceptibility of a population of HCV infecting a patient to the HCV inhibitor. In certain embodiments, the method comprises culturing a host cell comprising a plurality of patient-derived segments from the HCV population and an indicator gene in the presence of the HCV inhibitor, measuring the activity of the indicator gene in the host cell; and comparing the activity of the indicator gene as measured (by IC₅₀, IC₉₅, or ratio thereof, or slope or maximum inhibition percentage) with a reference activity of the indicator gene, wherein the difference between the measured activity of the indicator gene relative to the reference activity correlates with the susceptibility of the HCV to the HCV inhibitor, thereby determining the susceptibility of the HCV to the HCV inhibitor. In certain embodiments, the activity of the indicator gene depends on the activity of a plurality of polypeptide encoded by the plurality of patient-derived segments. In certain embodiments, the patient-derived segment comprises a nucleic acid sequence that encodes NS5B, NS5A, or NS3. In certain embodiments, the plurality of patient-derived segments is prepared by amplifying the patient-derived segments from a plurality of nucleic acids obtained from a sample from the patient.

In yet another aspect, the present invention provides a method for determining the susceptibility of a population of HCV infecting a patient to the HCV inhibitor. In certain embodiments, the method comprises culturing a host cell comprising a plurality of patient-derived segments obtained from the population of HCV and an indicator gene in the presence of varying concentrations of the HCV inhibitor, measuring the activity of the indicator gene in the host cell for the varying concentrations of the HCV inhibitor; and determining the IC₅₀, IC₉₅, or ratio thereof of the population of HCV to the anti-viral drug, wherein the IC₅₀, IC₉₅, or ratio thereof of the population of HCV to the HCV inhibitor indicates the susceptibility of the population of HCV to the HCV inhibitor. In certain embodiments, the host cell comprises a patient-derived segment and an indicator gene. In certain embodiments, the activity of the indicator gene depends on the activity of a plurality of polypeptides encoded by the plurality of patient-derived segments. In certain embodiments, the plurality of patient-derived segments comprises a nucleic acid sequence that encodes NS5B, NS5A, or NS3. In certain embodiments, the IC₅₀, IC₉₅, or ratio thereof of the population of HCV can be determined by plotting the activity of the indicator gene observed versus the log of anti-HCV drug concentration. In certain embodiments, the plurality of patient-derived segments is prepared by amplifying the patient-derived segments from a plurality of nucleic acids obtained from a sample from the patient. In certain other embodiments, the susceptibility of the HCV to the HCV inhibitor is determined by comparing the slope or maximum inhibition of the HCV identified in the curve to the curve of a reference virus.

Construction of a Resistance Test Vector

In certain embodiments, the resistance test vector can be made by insertion of a patient-derived segment into an indicator gene viral vector. Generally, in such embodiments, the resistance test vectors do not comprise all genes necessary to produce a fully infectious viral particle. In certain embodiments, the resistance test vector can be made by insertion of a patient-derived segment into a packaging vector while the indicator gene is contained in a second vector, for example an indicator gene viral vector. In certain embodiments, the resistance test vector can be made by insertion of a patient-derived segment into a packaging vector while the indicator gene is integrated into the genome of the host cell to be infected with the resistance test vector.

If a drug were to target more than one functional viral sequence or viral gene product, patient-derived segments comprising each functional viral sequence or viral gene product can be introduced into the resistance test vector. In the case of combination therapy, where two or more anti-HCV drugs targeting the same or two or more different functional viral sequences or viral gene products are being evaluated, patient-derived segments comprising each such functional viral coding sequence or viral gene product can be inserted in the resistance test vector. The patient-derived segments can be inserted into unique restriction sites or specified locations, called patient sequence acceptor sites, in the indicator gene viral vector or for example, a packaging vector depending on the particular construction selected

Patient-derived segments can be incorporated into resistance test vectors using any of suitable cloning technique known by one of skill in the art without limitation. For example, cloning via the introduction of class II restriction sites into both the plasmid backbone and the patient-derived segments, which is preferred, or by uracil DNA glycosylase primer cloning.

The patient-derived segment may be obtained by any method of molecular cloning or gene amplification, or modifications thereof, by introducing patient sequence acceptor sites, as described below, at the ends of the patient-derived segment to be introduced into the resistance test vector. In a preferred embodiment, a gene amplification method such as PCR can be used to incorporate restriction sites corresponding to the patient-sequence acceptor sites at the ends of the primers used in the PCR reaction. Similarly, in a molecular cloning method such as cDNA cloning, the restriction sites can be incorporated at the ends of the primers used for first or second strand cDNA synthesis, or in a method such as primer-repair of DNA, whether cloned or uncloned DNA, the restriction sites can be incorporated into the primers used for the repair reaction. The patient sequence acceptor sites and primers can be designed to improve the representation of patient-derived segments. Sets of resistance test vectors having designed patient sequence acceptor sites allows representation of patient-derived segments that could be underrepresented in one resistance test vector alone.

Resistance test vectors can be prepared by modifying an indicator gene viral vector by introducing patient sequence acceptor sites, amplifying or cloning patient-derived segments and introducing the amplified or cloned sequences precisely into indicator gene viral vectors at the patient sequence acceptor sites. In certain embodiments, the resistance test vectors can be constructed from indicator gene viral vectors, which in turn can be derived from genomic viral vectors or subgenomic viral vectors and an indicator gene cassette, each of which is described below. Resistance test vectors can then be introduced into a host cell. Alternatively, in certain embodiments, a resistance test vector can be prepared by introducing patient sequence acceptor sites into a packaging vector, amplifying or cloning patient-derived segments and inserting the amplified or cloned sequences precisely into the packaging vector at the patient sequence acceptor sites and co-transfecting this packaging vector with an indicator gene viral vector.

In one preferred embodiment, the resistance test vector may be introduced into packaging host cells together with packaging expression vectors, as defined below, to produce resistance test vector viral particles that are used in drug resistance and susceptibility tests that are referred to herein as a “particle-based test.” In an alternative embodiment, the resistance test vector may be introduced into a host cell in the absence of packaging expression vectors to carry out a drug resistance and susceptibility test that is referred to herein as a “non-particle-based test.” As used herein a “packaging expression vector” provides the factors, such as packaging proteins (e.g., structural proteins such as core and envelope polypeptides), transacting factors, or genes required by replication-defective HCV. In such a situation, a replication-competent viral genome is enfeebled in a manner such that it cannot replicate on its own. This means that, although the packaging expression vector can produce the trans-acting or missing genes required to rescue a defective viral genome present in a cell containing the enfeebled genome, the enfeebled genome cannot rescue itself. Such embodiments are particularly useful for preparing viral particles that comprise resistance test vectors which do not comprise all viral genes necessary to produce a fully infectious viral particle.

In certain embodiments, the resistance test vectors comprise an indicator gene, though as described above, the indicator gene need not necessarily be present in the resistance test vector. Examples of indicator genes include, but are not limited to, the E. coli lacZ gene which encodes beta-galactosidase, the luc gene which encodes luciferase either from, for example, Photonis pyralis (the firefly) or Renilla reniformis (the sea pansy), the E. coli phoA gene which encodes alkaline phosphatase, green fluorescent protein and the bacterial CAT gene which encodes chloramphenicol acetyltransferase. A preferred indicator gene is firefly luciferase. Additional examples of indicator genes include, but are not limited to, secreted proteins or cell surface proteins that are readily measured by assay, such as radioimmunoassay (RIA), or fluorescent activated cell sorting (FACS), including, for example, growth factors, cytokines and cell surface antigens (e.g. growth hormone, Il-2 or CD4, respectively). Still other exemplary indicator genes include selection genes, also referred to as selectable markers. Examples of suitable selectable markers for mammalian cells are dihydrofolate reductase (DHFR), thymidine kinase, hygromycin, neomycin, zeocin or E. coli gpt. In the case of the foregoing examples of indicator genes, the indicator gene and the patient-derived segment are discrete, i.e. distinct and separate genes. In some cases, a patient-derived segment may also be used as an indicator gene. In one such embodiment in which the patient-derived segment corresponds to one or more HCV genes which is the target of an anti-HCV agent, one of the HCV genes may also serve as the indicator gene. For example, a viral protease gene may serve as an indicator gene by virtue of its ability to cleave a chromogenic substrate or its ability to activate an inactive zymogen which in turn cleaves a chromogenic substrate, giving rise in each case to a color reaction.

As discussed above, a resistance test vector can be assembled from an indicator gene viral vector. As used herein, “indicator gene viral vector” refers to a vector(s) comprising an indicator gene and its control elements and one or more viral genes or coding regions. The indicator gene viral vector can be assembled from an indicator gene cassette and a “viral vector,” defined below. The indicator gene viral vector may additionally include an enhancer, splicing signals, polyadenylation sequences, transcriptional terminators, or other regulatory sequences. Additionally the indicator gene in the indicator gene viral vector may be functional or nonfunctional. In the event that the viral segments which are the target of the anti-viral drug are not included in the indicator gene viral vector, they can be provided in a second vector. An “indicator gene cassette” comprises an indicator gene and control elements, and, optionally, is configured with restriction enzyme cleavage sites at its ends to facilitate introduction of the cassette into a viral vector. A “viral vector” refers to a vector comprising some or all of the following: viral genes encoding a gene product, control sequences, viral packaging sequences, and in the case of a retrovirus, integration sequences. The viral vector may additionally include one or more viral segments, one or more of which may be the target of an anti-viral drug. Two examples of a viral vector which contain viral genes are referred to herein as an “genomic viral vector” and a “subgenomic viral vector.” A “genomic viral vector” is a vector which may comprise a deletion of a one or more viral genes to render the virus replication incompetent, e.g., unable to express all of the proteins necessary to produce a fully infectious viral particle, but which otherwise preserves the mRNA expression and processing characteristics of the complete virus. In one embodiment for an HCV drug susceptibility and resistance test, the genomic viral vector comprises the NS5B, NS5A, and NS3 coding regions. A “subgenomic viral vector” refers to a vector comprising the coding region of one or more viral genes which may encode the proteins that are the target(s) of the anti-viral drug. In a preferred embodiment, a subgenomic viral vector comprises the HCV polymerase coding region, or a portion thereof. In certain embodiments, the viral coding genes can be under the control of a native enhancer/promoter. In certain embodiments, the viral coding regions can be under the control of a foreign viral or cellular enhancer/promoter. In a preferred embodiment, the genomic or subgenomic viral coding regions can be under the control of the native enhancer/promoter region or the CMV immediate-early (IE) enhancer/promoter. In certain embodiments of an indicator gene viral vector that contains one or more viral genes which are the targets or encode proteins which are the targets of one or more anti-viral drug(s), the vector can comprise patient sequence acceptor sites. The patient-derived segments can be inserted in the patient sequence acceptor site in the indicator gene viral vector which is then referred to as the resistance test vector, as described above.

“Patient sequence acceptor sites” are sites in a vector for insertion of patient-derived segments. In certain embodiments, such sites may be: 1) unique restriction sites introduced by site-directed mutagenesis into a vector; 2) naturally occurring unique restriction sites in the vector; or 3) selected sites into which a patient-derived segment may be inserted using alternative cloning methods (e.g. UDG cloning). In certain embodiments, the patient sequence acceptor site is introduced into the indicator gene viral vector by site-directed mutagenesis. The patient sequence acceptor sites can be located within or near the coding region of the viral protein which is the target of the anti-viral drug. The viral sequences used for the introduction of patient sequence acceptor sites are preferably chosen so that no change is made in the amino acid coding sequence found at that position. If a change is made in the amino acid coding sequence at the position, the change is preferably a conservative change. Preferably the patient sequence acceptor sites can be located within a relatively conserved region of the viral genome to facilitate introduction of the patient-derived segments. Alternatively, the patient sequence acceptor sites can be located between functionally important genes or regulatory sequences. Patient-sequence acceptor sites may be located at or near regions in the viral genome that are relatively conserved to permit priming by the primer used to introduce the corresponding restriction site into the patient-derived segment. To improve the representation of patient-derived segments further, such primers may be designed as degenerate pools to accommodate viral sequence heterogeneity, or may incorporate residues such as deoxyinosine (I) which have multiple base-pairing capabilities. Sets of resistance test vectors having patient sequence acceptor sites that define the same or overlapping restriction site intervals may be used together in the drug resistance and susceptibility tests to provide representation of patient-derived segments that contain internal restriction sites identical to a given patient sequence acceptor site, and would thus be underrepresented in either resistance test vector alone.

Construction of the vectors of the invention employs standard ligation and restriction techniques which are well understood in the art. See, for example, Ausubel et al, 2005, Current Protocols in Molecular Biology Wiley—Interscience and Sambrook et al, 2001, Molecular Cloning: A Laboratory Manual, Cold Spring Harbor Laboratory, N.Y. Isolated plasmids, DNA sequences, or synthesized oligonucleotides can be cleaved, tailored, and relegated in the form desired. The sequences of all DNA constructs incorporating synthetic DNA can be confirmed by DNA sequence analysis. See, for example, Sanger et al, 1977, P.N.A.S. USA 74:5463-5467.

In addition to the elements discussed above, the vectors used herein may also contain a selection gene, also termed a selectable marker. In certain embodiments, the selection gene encodes a protein, necessary for the survival or growth of a host cell transformed with the vector. Examples of suitable selectable markers for mammalian cells include the dihydrofolate reductase gene (DHFR), the ornithine decarboxylase gene, the multi-drug resistance gene (mdr), the adenosine deaminase gene, and the glutamine synthase gene. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. The second category is referred to as dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (see Southern and Berg, 1982, J. Molec. Appl. Genet. 1:327, mycophenolic acid (see Mulligan and Berg, 1980, Science 209:1422, or hygromycin (see Sugden et al., 1985, Mol. Cell. Biol. 5:410-413. The three examples given above employ bacterial genes under eukaryotic control to convey resistance to the appropriate drug neomycin (G418 or genticin), xgpt (mycophenolic acid) or hygromycin, respectively.

Host Cells

In certain embodiments, the methods of the invention comprise culturing a host cell that comprises a patient-derived segment and an indicator gene. In certain embodiments, the host cells can be mammalian cells. In certain embodiments, the host cells can be derived from human tissues and cells which are the principle targets of viral infection. Human-derived host cells allow the anti-viral drug to enter the cell efficiently and be converted by the cellular enzymatic machinery into the metabolically relevant form of the anti-viral inhibitor. In some embodiments, host cells can be referred to herein as a “packaging host cells,” “resistance test vector host cells,” or “target host cells.” A “packaging host cell” refers to a host cell that provides the transacting factors and viral packaging proteins required by the replication defective viral vectors used herein, such as, e.g., the resistance test vectors, to produce resistance test vector viral particles. The packaging proteins may provide for expression of viral genes contained within the resistance test vector itself a packaging expression vector(s), or both. A packaging host cell can be a host cell which is transfected with one or more packaging expression vectors and when transfected with a resistance test vector is then referred to herein as a “resistance test vector host cell” and is sometimes referred to as a packaging host cell/resistance test vector host cell.

In certain embodiments, the host cell is a Huh7 cell. In other embodiments, the host cell is a Huh7 derivative (e.g., Huh7.5, Huh7.5.1). Huh7.5 cells—human hepatocyte cell line was generated by curing a stably selected HCV replicon-containing cell line with IFN. (Blight K J, et al. J Virol 76: 13001-13014, 2002). In certain other embodiments, the host cell is a HepG2 cell, a Hep3B cell, or a derivative thereof. In certain embodiments, the host cell is derived from a human hepatoma cell line. In certain embodiments, the host cell is a primary hepatocyte (e.g., from fetal, adult, or regenerating liver). In yet other embodiments, the host cell is a lymphocyte cell (e.g., B cell, B cell lymphoma).

Unless otherwise provided, the method used herein for transformation of the host cells is the calcium phosphate co-precipitation method of Graham and van der Eb, 1973, Virology 52:456-457. Alternative methods for transfection include, but are not limited to, electroporation, the DEAE-dextran method, lipofection and biolistics. See, e.g., Kriegler, 1990, Gene Transfer and Expression: A Laboratory Manual, Stockton Press.

Host cells may be transfected with the expression vectors of the present invention and cultured in conventional nutrient media modified as is appropriate for inducing promoters, selecting transformants or amplifying genes. Host cells are cultured in F12: DMEM (Gibco) 50:50 with added glutamine and without antibiotics. The culture conditions, such as temperature, pH and the like, are those previously used with the host cell selected for expression, and will be apparent to the ordinarily skilled artisan.

Drug Susceptibility and Resistance Tests

Drug susceptibility and resistance tests may be carried out in one or more host cells. Viral drug susceptibility is determined as the concentration of the anti-viral agent at which a given percentage of indicator gene expression is inhibited (e.g., the IC₅₀ for an anti-viral agent is the concentration at which 50% of indicator gene expression is inhibited). A standard curve for drug susceptibility of a given anti-viral drug can be developed for a viral segment that is either a standard laboratory viral segment or from a drug-naive patient (i.e., a patient who has not received any anti-viral drug) using the method of this invention. Correspondingly, viral drug resistance can be determined by detecting a decrease in viral drug susceptibility for a given patient either by comparing the drug susceptibility to such a given standard or by making sequential measurement in the same patient over time, as determined by increased inhibition of indicator gene expression (i.e. decreased indicator gene expression).

In certain embodiments, resistance test vector viral particles are produced by a first host cell (the resistance test vector host cell) that is prepared by transfecting a packaging host cell with the resistance test vector and packaging expression vector(s). The resistance test vector viral particles can then be used to infect a second host cell (the target host cell) in which the expression of the indicator gene is measured. Such a two cell system comprising a packaging host cell which is transfected with a resistance test vector, which is then referred to as a resistance test vector host cell, and a target cell are used in the case of either a functional or non-functional indicator gene. Functional indicator genes are efficiently expressed upon transfection of the packaging host cell, and thus infection of a target host cell with resistance test vector host cell supernatant is needed to accurately determine drug susceptibility. Non-functional indicator genes with a permuted promoter, a permuted coding region, or an inverted intron are not efficiently expressed upon transfection of the packaging host cell and thus the infection of the target host cell can be achieved either by co-cultivation by the resistance test vector host cell and the target host cell or through infection of the target host cell using the resistance test vector host cell supernatant.

In a second type of drug susceptibility and resistance test, a single host cell (the resistance test vector host cell) also serves as a target host cell. The packaging host cells are transfected and produce resistance test vector viral particles and some of the packaging host cells also become the target of infection by the resistance test vector particles. Drug susceptibility and resistance tests employing a single host cell type are possible with viral resistance test vectors comprising a non-functional indicator gene with a permuted promoter, a permuted coding region, or an inverted intron. Such indicator genes are not efficiently expressed upon transfection of a first cell, but are only efficiently expressed upon infection of a second cell, and thus provide an opportunity to measure the effect of the anti-viral agent under evaluation. In the case of a drug susceptibility and resistance test using a resistance test vector comprising a functional indicator gene, neither the co-cultivation procedure nor the resistance and susceptibility test using a single cell type can be used for the infection of target cells. A resistance test vector comprising a functional indicator gene can use a two cell system using filtered supernatants from the resistance test vector host cells to infect the target host cell.

In certain embodiments, a particle-based resistance tests can be carried out with resistance test vectors derived from genomic viral vectors, which can be cotransfected with a packaging expression vector. Alternatively, a particle-based resistance test may be carried out with resistance test vectors derived from subgenomic viral vectors which are cotransfected with a packaging expression vector. In another embodiment of the invention, non-particle-based resistance tests can be carried out using each of the above described resistance test vectors by transfection of selected host cells in the absence of packaging expression vectors.

In the case of the particle-based susceptibility and resistance test, resistance test vector viral particles can be produced by a first host cell (the resistance test vector host cell), that can be prepared by transfecting a packaging host cell with the resistance test vector and packaging expression vector(s) as described above. The resistance test vector viral particles can then be used to infect a second host cell (the target host cell) in which the expression of the indicator gene is measured. In a second type of particle-based susceptibility and resistance test, a single host cell type (the resistance test vector host cell) serves both purposes: some of the packaging host cells in a given culture can be transfected and produce resistance test vector viral particles and some of the host cells in the same culture can be the target of infection by the resistance test vector particles thus produced. Resistance tests employing a single host cell type are possible with resistance test vectors comprising a non-functional indicator gene with a permuted promoter since such indicator genes can be efficiently expressed upon infection of a permissive host cell, but are not efficiently expressed upon transfection of the same host cell type, and thus provide an opportunity to measure the effect of the anti-viral agent under evaluation. For similar reasons, resistance tests employing two cell types may be carried out by co-cultivating the two cell types as an alternative to infecting the second cell type with viral particles obtained from the supernatants of the first cell type.

In the case of the non-particle-based susceptibility and resistance test, resistance tests can be performed by transfection of a single host cell with the resistance test vector in the absence of packaging expression vectors. Non-particle based resistance tests can be carried out using the resistance test vectors comprising non-functional indicator genes with either permuted promoters, permuted coding regions or inverted introns. These non-particle based resistance tests are performed by transfection of a single host cell type with each resistance test vector in the absence of packaging expression vectors. Although the non-functional indicator genes contained within these resistance test vectors are not efficiently expressed upon transfection of the host cells, there is detectable indicator gene expression resulting from non-viral particle-based reverse transcription. Reverse transcription and strand transfer results in the conversion of the permuted, non-functional indicator gene to a non-permuted, functional indicator gene. As reverse transcription is completely dependent upon the expression of the polymerase gene contained within each resistance test vector, anti-viral agents may be tested for their ability to inhibit the polymerase gene product, encoded by the patient-derived segments contained within the resistance test vectors.

The packaging host cells can be transfected with the resistance test vector and the appropriate packaging expression vector(s) to produce resistance test vector host cells. In certain embodiments, individual anti-viral agents, can be added to individual plates of packaging host cells at the time of their transfection, at an appropriate range of concentrations. Twenty-four to 48 hours after transfection, target host cells can be infected by co-cultivation with resistance test vector host cells or with resistance test vector viral particles obtained from filtered supernatants of resistance test vector host cells. Each anti-viral agent, or combination thereof, can be added to the target host cells prior to or at the time of infection to achieve the same final concentration of the given agent, or agents, present during the transfection. In other embodiments, the anti-viral agent(s) can be omitted from the packaging host cell culture, and added only to the target host cells prior to or at the time of infection.

Determination of the expression or inhibition of the indicator gene in the target host cells infected by co-cultivation or with filtered viral supernatants can be performed measuring indicator gene expression or activity. For example, in the case where the indicator gene is the firefly luc gene, luciferase activity can be measured. The reduction in luciferase activity observed for target host cells infected with a given preparation of resistance test vector viral particles in the presence of a given antiviral agent, or agents, as compared to a control run in the absence of the antiviral agent, generally relates to the log of the concentration of the antiviral agent as a sigmoidal curve. This inhibition curve can be used to calculate the apparent inhibitory concentration (IC) of that agent, or combination of agents, for the viral target product encoded by the patient-derived segments present in the resistance test vector.

In the case of a one cell susceptibility and resistance test, host cells can be transfected with the resistance test vector and the appropriate packaging expression vector(s) to produce resistance test vector host cells. Individual antiviral agents, or combinations thereof can be added to individual plates of transfected cells at the time of their transfection, at an appropriate range of concentrations. Twenty-four to 72 hours after transfection, cells can be collected and assayed for indicator gene, e.g., firefly luciferase, activity. As transfected cells in the culture do not efficiently express the indicator gene, transfected cells in the culture, as well superinfected cells in the culture, can serve as target host cells for indicator gene expression. The reduction in luciferase activity observed for cells transfected in the presence of a given antiviral agent, or agents as compared to a control run in the absence of the antiviral agent(s), generally relates to the log of the concentration of the antiviral agent as a sigmoidal curve. This inhibition curve can be used to calculate the apparent inhibitory concentration (IC), slope, and/or maximum inhibition percentage of an agent, or combination of agents, for the viral target product encoded by the patient-derived segments present in the resistance test vector.

Antiviral Drugs/Drug Candidates

The antiviral drugs being added to the test system can be added at selected times depending upon the target of the antiviral drug. In certain embodiments, the HCV inhibitor is a nucleos(t)ide inhibitor (NI) or protease inhibitor (PI). In certain embodiments, the HCV inhibitor is an interferon. In some embodiments, the interferon may comprise, for example, pegylated interferon alpha 2a, pegylated interferon alpha 2b, pegylated interferon lambda, parentals or derivatives of the above, or any member of the interferon family or derivative thereof with activity against HCV. In other embodiments, the HCV inhibitor is a non-nucleoside inhibitor (NNI). In some embodiments, the HCV inhibitor is an NNI that targets site A, B, C, or D of the HCV polymerase (NNI-A, NNI-B, NNI-C, or NNI-D). The HCV inhibitor may be, in some embodiments, NS3-targeting (e.g., BILN-2061, VX-950, SCH-503,034, SCH-900,518, TMC-435,350, R-7227 (ITMN-191), MK-5172, MK-7009, BI-201,335, BMS-650,032, BMS-824,393, PHX-1766, ACH-1625, ACH-2684, VX-985, BMS-791,325, IDX-320, GS-9256, GS-9451, ABT-450, VX-500, BIT-225), NS5A-targeting (e.g., BMS-790,052, GSK-2336805, PPI-461, ABT-267, GS-5885, ACH-2928, AZD-7295), or NS5B-targeting (e.g., NM-283, RG-7128, R-1626, PSI-7851, IDX-184, MK-0608, PSI-7977, PSI-938, GS-6620, TMC-649,128, INX-189, VX-759, VCH-916, VX-222, ANA-598, HCV-796, GS-9190, GS-9669, ABT-333, PF-4878691, IDX-375, ABT-837,093, GSK-625,443, ABT-072), as well as combinations thereof, and can be added to individual plates of target host cells at the time of infection by the resistance test vector viral particles, at a test concentration. Alternatively, the antiviral drugs may be present throughout the assay. The test concentration is selected from a range of concentrations which is typically between about 0.1 nM and about 100 μM, between about 1 nM and about 100 μM, between about 10 nM and about 100 μM, between about 0.1 nM and about 10 μM, between about 1 nM and about 10 μM, between about 10 nM and about 100 μM, between about 0.1 nM and about 1 μM, between about 1 nM and about 1 μM, or between about 0.01 nM and about 0.1 μM.

In certain embodiments, a candidate antiviral compound can be tested in a drug susceptibility test of the invention. The candidate antiviral compound can be added to the test system at an appropriate concentration and at selected times depending upon the protein target of the candidate anti-viral. Alternatively, more than one candidate antiviral compound may be tested or a candidate antiviral compound may be tested in combination with an antiviral drug. The effectiveness of the candidate antiviral compound can be evaluated by measuring the activity of the indicator gene. If the candidate compound is effective at inhibiting a viral polypeptide activity, the activity of the indicator gene will be reduced in the presence of the candidate compound relative to the activity observed in the absence of the candidate compound. In another aspect of this embodiment, the drug susceptibility and resistance test may be used to screen for viral mutants. Following the identification of resistant mutants to either known anti-viral drugs or candidate anti-viral drugs the resistant mutants can be isolated and the DNA analyzed. A library of viral resistant mutants can thus be assembled enabling the screening of candidate anti-viral agents, either alone or in combination with other known or putative anti-viral agents.

Methods of Determining Replication Capacity of an HCV

In another aspect, the invention provides a method for determining the replication capacity of a hepatitis C virus (HCV). In certain embodiments, the method comprises culturing a host cell comprising a patient-derived segment and an indicator gene, measuring the activity of the indicator gene in the host cell, wherein the activity of the indicator gene between the activity of the indicator gene measured relative to a reference activity indicates the replication capacity of the HCV, thereby determining the replication capacity of the HCV. In certain embodiments, the activity of the indicator gene depends on the activity of a polypeptide encoded by the patient-derived segment. In certain embodiments, the patient-derived segment comprises a nucleic acid sequence that encodes NS5B, NS3, and/or NS5A.

In certain embodiments, the reference activity of the indicator gene is an amount of activity determined by performing a method of the invention with a standard laboratory viral segment. In certain embodiments, the standard laboratory viral segment comprises a nucleic acid sequence from HCV strain Con1 or H77. In other embodiments, the reference viral segment is a nucleic acid sequence from the patient HCV prior to treatment with an inhibitor.

In certain embodiments, the HCV is determined to have increased replication capacity relative to the reference. In certain embodiments, the HCV is determined to have reduced replication capacity relative to the reference. In certain embodiments, the host cell is a Huh7 cell. In certain embodiments, the patient-derived segment encodes NS5B, NS3, and/or NS5A.

In certain embodiments, the phenotypic analysis can be performed using recombinant virus assays (“RVAs”). In certain embodiments, RVAs use virus stocks generated by homologous recombination or between viral vectors and viral gene sequences, amplified from the patient virus. In certain embodiments, RVAs virus stocks generated by ligating viral gene sequences, amplified from patient virus, into viral vectors. In certain embodiments, the patient-derived segment encodes NS5B, NS3, and/or NS5A.

The methods of determining replication capacity can be used, for example, with nucleic acids from amplified viral gene sequences. As discussed below, the nucleic acid can be amplified from any sample known by one of skill in the art to contain a viral gene sequence, without limitation. For example, the sample can be a sample from a human or an animal infected with the virus or a sample from a culture of viral cells. In certain embodiments, the viral sample comprises a genetically modified laboratory strain. In certain embodiments, the genetically modified laboratory strain comprises a site-directed mutation. In other embodiments, the viral sample comprises a wild-type isolate. In certain embodiments, the wild-type isolate is obtained from a treatment-naive patient. In certain embodiments, the wild-type isolate is obtained from a treatment-experienced patient.

A resistance test vector (“RTV”) can then be constructed by incorporating the amplified viral gene sequences into a replication defective viral vector by using any method known in the art of incorporating gene sequences into a vector. In one embodiment, restrictions enzymes and conventional cloning methods are used. See Sambrook et al, 2001, Molecular Cloning: A Laboratory Manual, Cold Spring Harbor Laboratory, 3.sup.rd ed., NY; and Ausubel et al., 1989, Current Protocols in Molecular Biology, Greene Publishing Associates and Wiley Interscience, NY. In a preferred embodiment, ApaI, PinAI, and XhoI restriction enzymes are used. Preferably, the replication defective viral vector is the indicator gene viral vector (“IGVV”). In a preferred embodiment, the viral vector contains a means for detecting replication of the RTV. Preferably, the viral vector comprises a luciferase gene.

The assay can be performed by first co-transfecting host cells with RTV DNA and a plasmid that expresses the envelope proteins of another virus, for example, amphotropic murine leukemia virus (MLV). Following transfection, viral particles can be harvested from the cell culture and used to infect fresh target cells in the presence of varying amounts of anti-viral drug(s). The completion of a single round of viral replication in the fresh target cells can be detected by the means for detecting replication contained in the vector. In a preferred embodiment, the means for detecting replication is an indicator gene. In a preferred embodiment, the indicator gene is firefly luciferase. In such preferred embodiments, the completion of a single round of viral replication results in the production of luciferase.

In certain embodiments, the HCV strain that is evaluated is a wild-type isolate of HCV. In other embodiments, the HCV strain that is evaluated is a mutant strain of HCV. In certain embodiments, such mutants can be isolated from patients. In other embodiments, the mutants can be constructed by site-directed mutagenesis or other equivalent techniques known to one of skill in the art. In still other embodiments, the mutants can be isolated from cell culture. The cultures can comprise multiple passages through cell culture in the presence of antiviral compounds to select for mutations that accumulate in culture in the presence of such compounds.

In one embodiment, viral nucleic acid, for example, HCV RNA is extracted from plasma samples, and a fragment of or entire viral coding regions can be amplified by methods such as, but not limited to PCR. See, e.g., Hertogs et al., 1998, Antimicrob. Agents Chemother. 42(2):269-76. In one example, a patient derived segment can be amplified by reverse transcription-PCR and then cotransfected into a host cell with a plasmid from which most of those sequences are deleted. Homologous recombination can then lead to the generation of chimeric viruses. The replication capacities of the chimeric viruses can be determined by any cell viability assay known in the art, and compared to replication capacities of a reference to assess whether a virus has altered replication capacity or is resistant or hypersusceptible to the antiviral drug. In certain embodiments, the reference can be the replication capacities of a statistically significant number of individual viral isolates. In other embodiments, the reference can be the replication capacity of a reference virus such as Con1 or H77. For example, an MT4 cell-3-(4,5-dimethylthiazol-2-yl)-2,5-diphenyltetrazolium bromide-based cell viability assay can be used in an automated system that allows high sample throughput.

Other assays for evaluating the phenotypic susceptibility of a virus to anti-viral drugs known to one of skill in the art can be adapted to determine replication capacity or to determine antiviral drug susceptibility or resistance.

One skilled in the art will recognize that the above-described methods for determining the replication capacity of an HCV can readily be adapted to perform methods for determining an HCV inhibitor susceptibility. Similarly, one of skill in the art will recognize that the above-described methods for determining inhibitor susceptibility can readily be adapted to perform methods for determining the replication capacity of an HCV. Adaptation of the methods for determining replication capacity can generally comprise performing the methods of the invention in the presence of varying concentration of antiviral drug. By doing so, the susceptibility of the HCV to the drug can be determined. Similarly, performing a method for determining inhibitor susceptibility in the absence of any antiviral drug can provide a measure of the replication capacity of the HCV used in the method.

### Computer-Implemented Methods for Determining Susceptibility or Replication Capacity

In another aspect, the present invention provides computer-implemented methods for determining the susceptibility of an HCV to an HCV inhibitor or determining the replication capacity of an HCV. In such embodiments, the methods of the invention are adapted to take advantage of the processing power of modern computers. One of skill in the art can readily adapt the methods in such a manner.

In certain embodiments, the invention provides a computer-implemented method for determining the susceptibility of an HCV to an HCV inhibitor. In certain embodiments, the method comprises inputting information regarding the activity of an indicator gene determined according to a method of the invention and a reference activity of an indicator gene and instructions to compare the activity of the indicator gene determined according to a method of the invention with the reference activity of the indicator gene into a computer memory; and comparing the activity of the indicator gene determined according to a method of the invention with the reference activity of the indicator gene in the computer memory, wherein the difference between the measured activity of the indicator gene relative to the reference activity correlates with the susceptibility of the HCV to the HCV inhibitor, thereby determining the susceptibility of the HCV to the HCV inhibitor.

In certain embodiments, the methods further comprise displaying the susceptibility of the HCV to the HCV inhibitor on a display of the computer. In certain embodiments, the methods further comprise printing the susceptibility of the HCV to the HCV inhibitor on a paper.

In another aspect, the invention provides a print-out indicating the susceptibility of the HCV to the HCV inhibitor determined according to a method of the invention. In still another aspect, the invention provides a computer-readable medium comprising data indicating the susceptibility of the HCV to the HCV inhibitor determined according to a method of the invention.

In another aspect, the invention provides a computer-implemented method for determining the replication capacity of an HCV. In certain embodiments, the method comprises inputting information regarding the activity of an indicator gene determined according to a method of the invention and a reference activity of an indicator gene and instructions to compare the activity of the indicator gene determined according to a method of the invention with the reference activity of the indicator gene into a computer memory; and comparing the activity of the indicator gene determined according to a method of the invention with the reference activity of the indicator gene in the computer memory, wherein the comparison of the measured activity of the indicator gene relative to the reference activity indicates the replication capacity of the HCV, thereby determining the replication capacity of the HCV.

In certain embodiments, the methods further comprise displaying the replication capacity of the HCV on a display of the computer. In certain embodiments, the methods further comprise printing the replication capacity of the HCV on a paper.

In another aspect, the invention provides a print-out indicating the replication capacity of the HCV, where the replication capacity is determined according to a method of the invention. In still another aspect, the invention provides a computer-readable medium comprising data indicating the replication capacity of the HCV, where the replication capacity is determined according to a method of the invention.

In still another aspect, the invention provides an article of manufacture that comprises computer-readable instructions for performing a method of the invention.

In yet another aspect, the invention provides a computer system that is configured to perform a method of the invention.

### Viruses and Viral Samples

Any virus known by one of skill in the art without limitation can be used as a source of patient-derived segments or viral sequences for use in the methods of the invention. In certain embodiments, the virus is an HCV and may be genotype 1, genotype 2, genotype 3, genotype 4, genotype 5, or genotype 6. In one embodiment of the invention, the virus is HCV genotype 1, 2, 3, or 4. In certain embodiments, the virus is HCV genotype 1a, 1b, 2a, or 2b.

Viruses from which patient-derived segments or viral gene sequences are obtained can be found in a viral sample obtained by any means known in the art for obtaining viral samples. Such methods include, but are not limited to, obtaining a viral sample from an individual infected with the virus or obtaining a viral sample from a viral culture. In one embodiment, the viral sample is obtained from a human individual infected with the virus. The viral sample could be obtained from any part of the infected individual's body or any secretion expected to contain the virus. Examples of such parts and secretions include, but are not limited to blood, serum, plasma, sputum, lymphatic fluid, semen, vaginal mucus, liver biopsy, and samples of other bodily fluids. In a preferred embodiment, the sample is a blood, serum, or plasma sample.

In another embodiment, a patient-derived segment or viral coding region sequence can be obtained from a virus that can be obtained from a culture. In some embodiments, the culture can be obtained from a laboratory. In other embodiments, the culture can be obtained from a collection, for example, the American Type Culture Collection.

In another embodiment, a patient-derived segment or viral coding region sequence can be obtained from a genetically modified virus. The virus can be genetically modified using any method known in the art for genetically modifying a virus. For example, the virus can be grown for a desired number of generations in a laboratory culture. In one embodiment, no selective pressure is applied (i.e., the virus is not subjected to a treatment that favors the replication of viruses with certain characteristics), and new mutations accumulate through random genetic drift. In another embodiment, a selective pressure is applied to the virus as it is grown in culture (i.e., the virus is grown under conditions that favor the replication of viruses having one or more characteristics). In one embodiment, the selective pressure is an anti-viral treatment. Any known anti-viral treatment can be used as the selective pressure.

In another aspect, the patient-derived segment or viral coding region sequence can be made by mutagenizing a virus, a viral genome, or a part of a viral genome. Any method of mutagenesis known in the art can be used for this purpose. In certain embodiments, the mutagenesis is essentially random. In certain embodiments, the essentially random mutagenesis is performed by exposing the virus, viral genome or part of the viral genome to a mutagenic treatment. In another embodiment, a coding region or gene that encodes a viral protein that is the target of an anti-viral therapy is mutagenized. Examples of essentially random mutagenic treatments include, for example, exposure to mutagenic substances (e.g., ethidium bromide, ethylmethanesulphonate, ethyl nitroso urea (ENU) etc.) radiation (e.g., ultraviolet light), the insertion and/or removal of transposable elements (e.g., Tn5, Tn10), or replication in a cell, cell extract, or in vitro replication system that has an increased rate of mutagenesis. See, e.g., Russell et al, 1979, Proc. Nat. Acad. Sci. USA 76:5918-5922; Russell, W., 1982, Environmental Mutagens and Carcinogens: Proceedings of the Third International Conference on Environmental Mutagens. One of skill in the art will appreciate that while each of these methods of mutagenesis is essentially random, at a molecular level, each has its own preferred targets.

In another aspect, the patient-derived segment or viral coding region sequence can be made using site-directed mutagenesis. Any method of site-directed mutagenesis known in the art can be used (see e.g., Sambrook et al., 2001, Molecular Cloning: A Laboratory Manual, Cold Spring Harbor Laboratory, 3rd ed., NY; and Ausubel et al, 2005, Current Protocols in Molecular Biology, Greene Publishing Associates and Wiley Interscience, NY, and Sarkar and Sommer, 1990, Biotechniques, 8:404-407). The site directed mutagenesis can be directed to, e.g., a particular coding region, gene, or genomic region, a particular part of a coding region, gene, or genomic region, or one or a few particular nucleotides within a coding region, gene, or genomic region. In one embodiment, the site directed mutagenesis is directed to a viral genomic region, coding region, gene, gene fragment, or nucleotide based on one or more criteria. In one embodiment, a coding region or gene, or a portion of a coding region or gene is subjected to site-directed mutagenesis because it encodes a protein that is known or suspected to be a target of an anti-viral therapy, e.g., the NS5B coding region encoding HCV RNA dependent RNA polymerase, or a portion thereof. In another embodiment, a portion of a coding region or gene, or one or a few nucleotides within a coding region or gene, are selected for site-directed mutagenesis. In one embodiment, the nucleotides to be mutagenized encode amino acid residues that are known or suspected to interact with an anti-viral compound. In another embodiment, the nucleotides to be mutagenized encode amino acid residues that are known or suspected to be mutated in viral strains that are resistant or susceptible or hypersusceptible to one or more antiviral agents. In another embodiment, the mutagenized nucleotides encode amino acid residues that are adjacent to or near in the primary sequence of the protein residues known or suspected to interact with an anti-viral compound or known or suspected to be mutated in viral strains that are resistant or susceptible or hypersusceptible to one or more antiviral agents. In another embodiment, the mutagenized nucleotides encode amino acid residues that are adjacent to or near to in the secondary, tertiary, or quaternary structure of the protein residues known or suspected to interact with an anti-viral compound or known or suspected to be mutated in viral strains having an altered replication capacity. In another embodiment, the mutagenized nucleotides encode amino acid residues in or near the active site of a protein that is known or suspected to bind to an anti-viral compound.

### EXAMPLES

### Example 1

### Preparation of Samples for Phenotypic Analysis

### Sample Preparation and Amplification

Most samples were received as frozen plasma and were accompanied by information including HCV genotype and/or subtype (e.g., 1a, 1b, 2, 3, 4) and viral load. Samples were thawed and stored in frozen aliquots if necessary, and a 200 μL aliquot was processed. Virus particles were disrupted by addition of lysis buffer containing a chaotropic agent. Genomic viral RNA (vRNA) was extracted from viral lysates using oligo-nucleotide linked magnetic beads. Purified vRNA was used as a template for first-strand cDNA synthesis in a reverse transcriptase (RT) reaction. The resulting cDNA was used as the template for the first round of a nested polymerase chain reaction (PCR) that results in the amplification of the entire NS5B region. Due to the sequence variation between different genotypes, specific RT and first and second round PCR primers were used. If genotype and/or subtype information was not available, more than one primer set can be used sequentially or in parallel.

Cloning Patient Derived Segment into the Resistance Test Vector

The second round (nested) PCR amplification primer set contained restriction endonuclease recognition/cleavage sites that enable cloning of NS5B amplification products into an HCV replicon resistance test vector (RTV) for phenotypic drug susceptibility analysis. PCR products were purified by agarose gel electrophoresis and subsequent column chromatography to remove residual primers, primer-dimers, and non-specific reaction products and were then subjected to restriction endonuclease digestion. The digestion reaction was purified using column chromatography, and the amplification product was then ligated into a luciferase reporter replicon RTV. Ligation reactions were used to transform competent E. coli. Plasmid DNA was purified from bacterial cultures, using silica column chromatography, and was quantified by spectrophotometry.

### Preparation of RTV RNA

Prior to in vitro transcription of the RTV, the plasmid DNA template was linearized by restriction endonuclease digestion and column purified. The RTV contains hepatitis delta virus ribozyme sequences for appropriate termination of replicon RNA following in vitro transcription. In vitro transcribed RNA was column purified, quantified, and the integrity was evaluated using electrophoretic separation.

### Example 2

### Phenotypic Assay for Determining HCV Inhibitor Susceptibility

RTV RNA was electroporated into a Huh7 cell line, and electroporated cells were incubated in the absence and presence of serially diluted inhibitors. RNA input was monitored by measuring the amount of luciferase activity produced in the electroporated cells at 4 hours post-electroporation. Luciferase activity is expressed as relative light units (RLU). Replication capacity (RC) was determined by evaluating luciferase activity at 72-96 hours postelectroporation in the absence of inhibitor, relative to RNA input and a control reference replicon RTV (Con1). A replication defective Con1 replicon (Con1 polymerase defective) was utilized to determine assay background (data not shown). Inhibitor susceptibility was determined by evaluating the ability of RTVs to replicate in the absence and presence of inhibitor at 72-96 hours post-electroporation. The % inhibition at each serial diluted inhibitor concentration was derived as follows:

[1−(luciferase activity in the presence of inhibitor luciferase activity in the absence of inhibitor)]×100

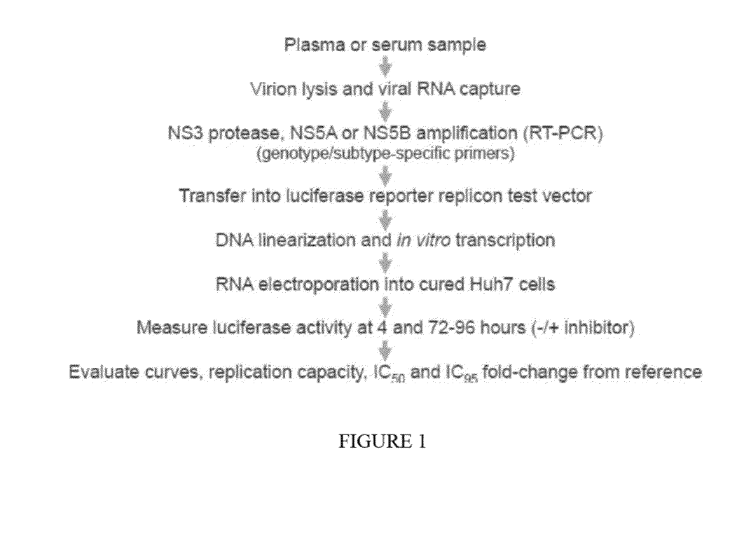

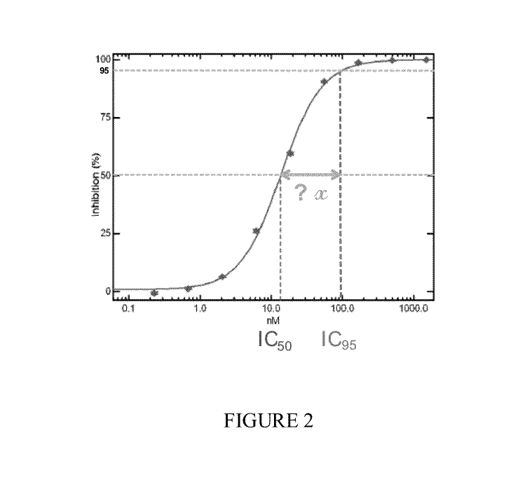

Inhibitor susceptibility profiles (curves) were derived from these values, and inhibition data (e.g., IC₅₀, the inhibitor concentration required to reduce virus replication by 50%; and IC₉₅, the inhibitor concentration required to reduce virus replication by 95%) was extrapolated from fitted curves. Inhibition data are reported as fold-change relative to that of a reference RTV (e.g., IC₅₀ (sample)/IC₅₀ (reference)) processed in the same assay batch (e.g., IC₅₀ fold-change (FC) from reference). An example of the PhenoSense® HCV NS5B Assay workflow is shown in FIG. 1, and a representative inhibitor susceptibly curve is shown in FIG. 2.

Assay accuracy was assessed by evaluating the HCV polymerase inhibitor susceptibility of RTVs containing the NS5B region of well-characterized subtype 1a (H77) and 1b (Con1) reference sequences and subtype 1a and 1b reference sequences engineered by site-directed mutagenesis (SDM) to contain mutations that confer reduced susceptibility to inhibitors of HCV RdRp (data not shown). Inhibitor susceptibility data (IC₅₀-FC and IC₉₅-FC) were analyzed for concordance with phenotypic data reported in the scientific literature. Replicons containing NS5B mutations exhibited expected reductions in susceptibility to nucleos(t)ide (NI; S282T mutants) and non-nucleoside polymerase inhibitors targeting site A (NNI-A; L392I and P495A/L mutants), site B (NNI-B; M423T), site C (NNI-C; C316Y and Y448H) and site D (NNI-D; C316Y), demonstrating assay accuracy (data not shown).

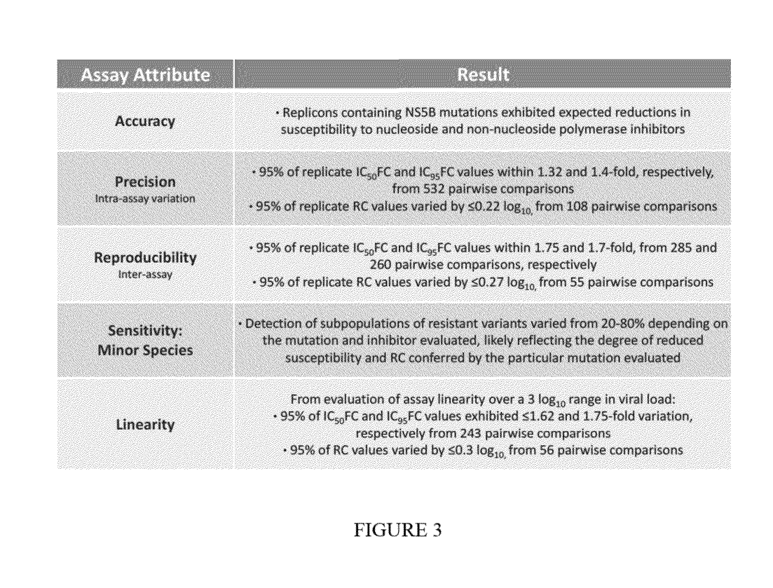

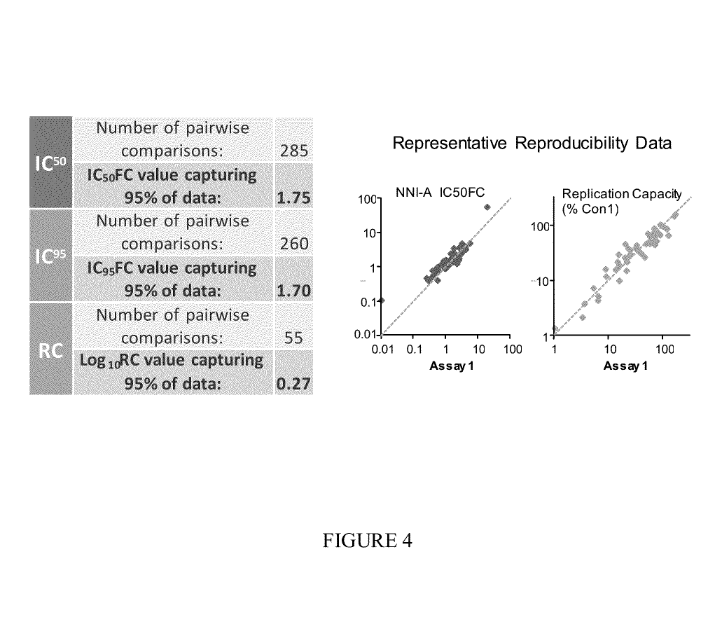

From analysis of intra-assay variation in inhibitor susceptibility measurements, 95% of replicate IC₅₀ FC and IC₉₅ FC values were within 1.32 and 1.4-fold, respectively, from 532 pairwise comparisons. 95% of replicate RC values varied by <0.22 log₁₀, based on 108 pairwise comparisons (FIG. 3). From analysis of inter-assay variation in inhibitor susceptibility measurements, 95% of replicate IC₅₀ FC and IC₉₅ FC values were within 1.75 and 1.7-fold, from 285 and 260 pairwise comparisons, respectively. 95% of replicate RC values varied by ≦0.27 log₁₀, based on 55 pairwise comparisons (FIGS. 3 and 4). The evaluation of assay linearity over a 3 log₁₀ range in viral load demonstrated that 95% of IC₅₀ FC and IC₉₅ FC values exhibited ≦1.62 and 1.75-fold variation, respectively from 243 pairwise comparisons. 95% of RC values varied by ≦0.3 log₁₀, based on 56 pairwise comparisons of serially diluted plasma samples (FIGS. 3 and 4).

### Example 3

### Measurement of IC₅₀ and IC₉₅ FC to Detect Susceptibility to RBV and NI

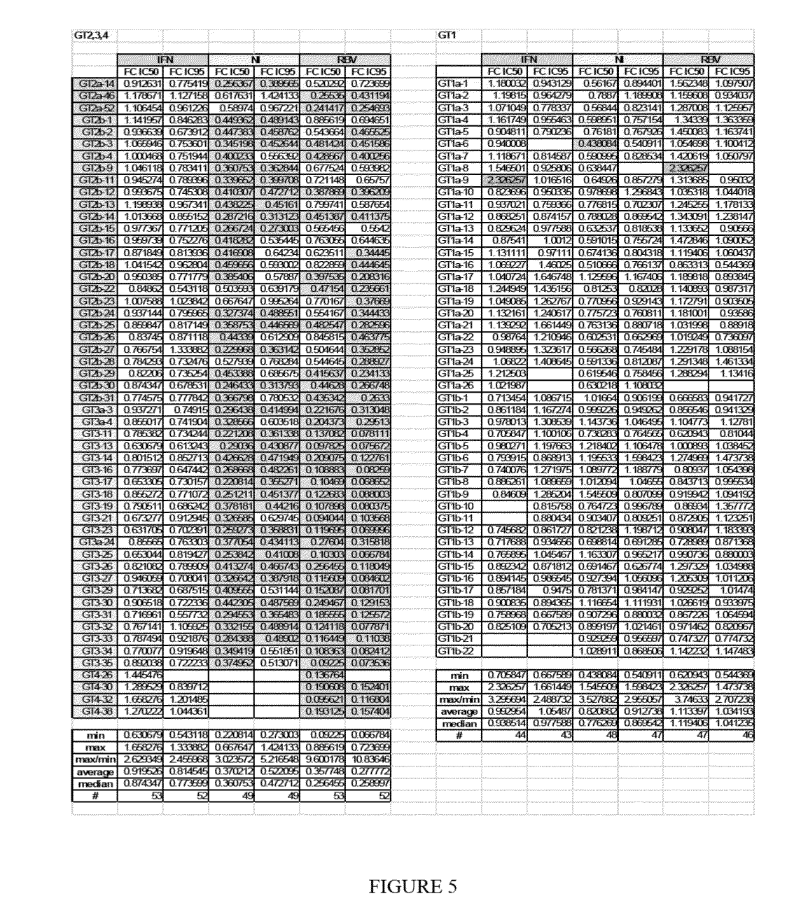

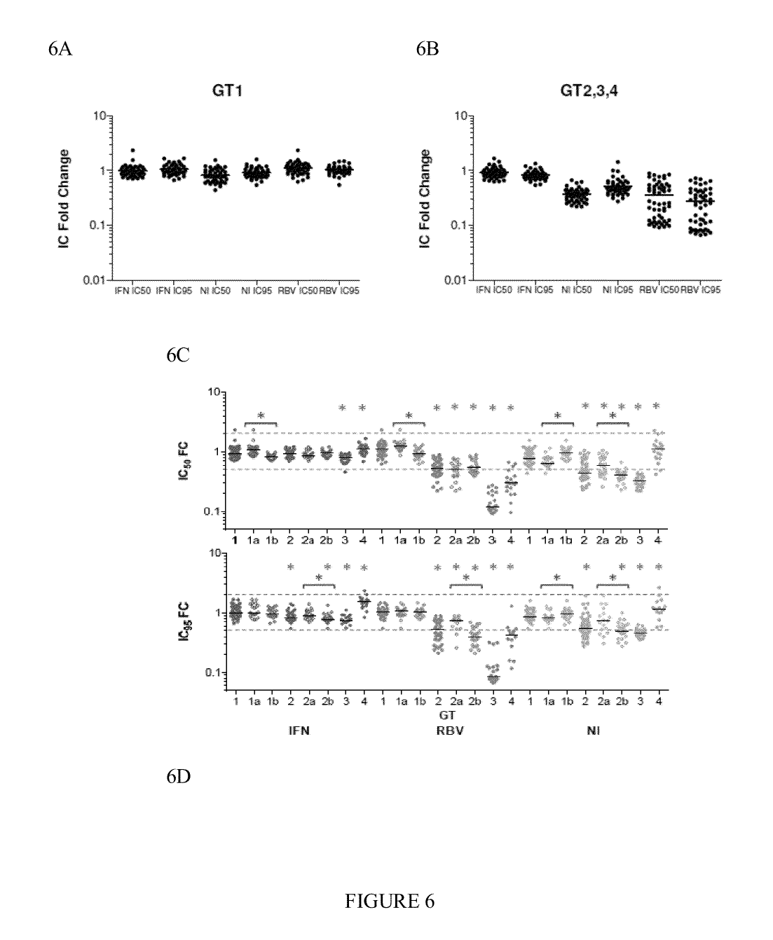

To evaluate the sensitivity of the PhenoSense® HCV NS5B Assay to detect sensitivity to ribavirin and nucleos(t)ide inhibitors, a panel of replicons were generated that contained patient-derived NS5B regions from GT1 (a/b), GT2 (a/b/k), GT3 (a/unknown), and GT4 (a/d/n/unknown) viruses. The various replicons were used in the phenotypic susceptibility assays described herein. The majority of replicons exhibited a replication capacity sufficient for evaluating inhibitor susceptibility (data not shown). Susceptibility to IFN, RBV, and an NI was tested to evaluate biological variation. The raw data is shown in the table in FIG. 5, and the data is shown graphically in FIG. 6. In FIG. 6, the inhibitor and its IC value are indicated on the x axis, and the IC fold change with respect to a reference virus (Con1 GT1b) is on the y axis.

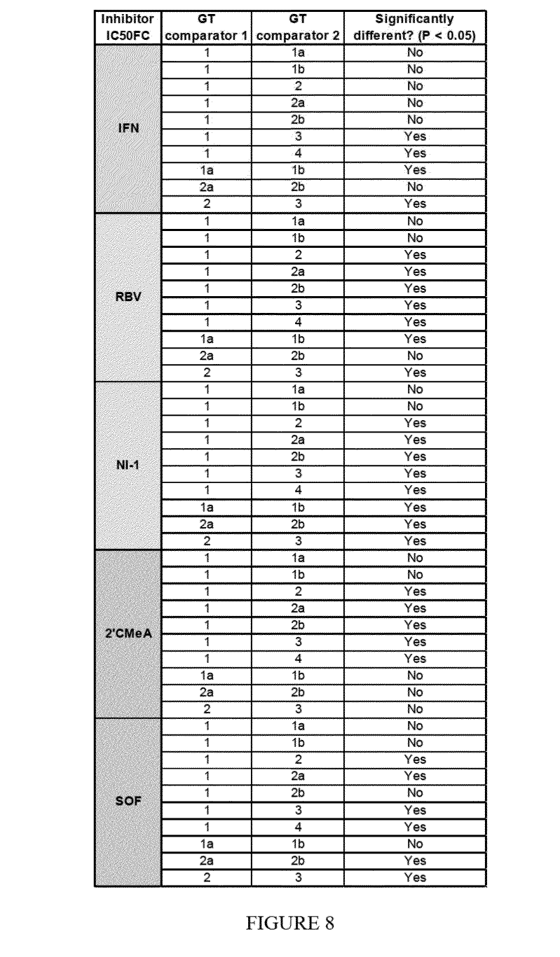

GT1, GT2, GT3, and GT4 chimeric replicons had similar susceptibilities to IFN (left most two groups in FIGS. 6A and 6B). Although the IFN susceptibilities are similar, there are small but significant differences as indicated in FIGS. 6C, 6D, and 8 (discussed below). GT1 replicons had similar susceptibilities to RBV and NI (right four groups of dots in FIG. 6A). Similarly, although the RBV and NI differences between GT1a and 1b viruses are similar, there are small but significant differences as indicated in FIGS. 6C, 6D, and 8. On whole, GT2, GT3, and GT4 replicons exhibited variation in RBV and/or NI susceptibility, with many viruses exhibiting increased susceptibility (up to approximately 10-fold) to RBV and/or NI (right four groups of dots in FIG. 6B). The data was further studied with respect to genotype subtypes as shown in FIGS. 6C and 6D. The IC₅₀ or IC₉₅ fold change, respectively, was plotted on the y axis, and the inhibitors and HCV genotype are plotted on the x axis.

A panel of HCV replicons containing patient-derived NS5B sequences from GT1-4 viruses was used to document variation in HCV inhibitor susceptibility. IFN susceptibility was similar within and between genotypes. RBV and NI susceptibility was similar among replicons with GT1a/b NS5B regions, but more variable between genotypes. A number of non-GT1 viruses exhibited relatively increased susceptibility to RBV and/or NI. This observation may contribute to improved SVR rates to RBV and/or NI containing regimens among patients infected with non-GT1 viruses compared to GT1 viruses. Accordingly, such information would be useful in determining the appropriate treatment regimen for a given individual.

These data also may be useful to inform clinical trial design, pre-treatment decisions (e.g., drugs to use, number of drugs to combine, treatment duration), as well as for evaluating resistance. In particular, phenotypic data, in conjunction with clinical outcome data, may further strengthen the utility of the assay e.g. for developing clinical cut offs.

### Example 4

### Measurement of IC₅₀ FC to Detect Inhibitor Susceptibility

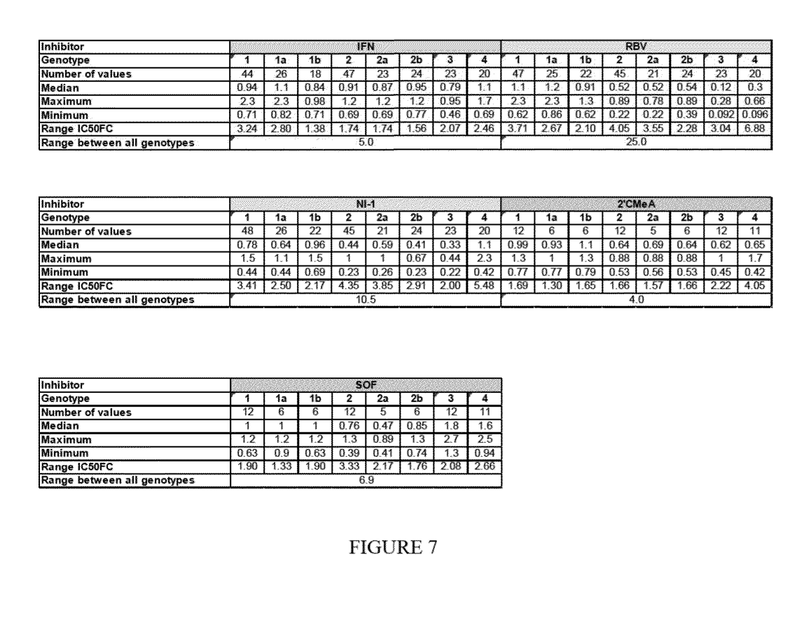

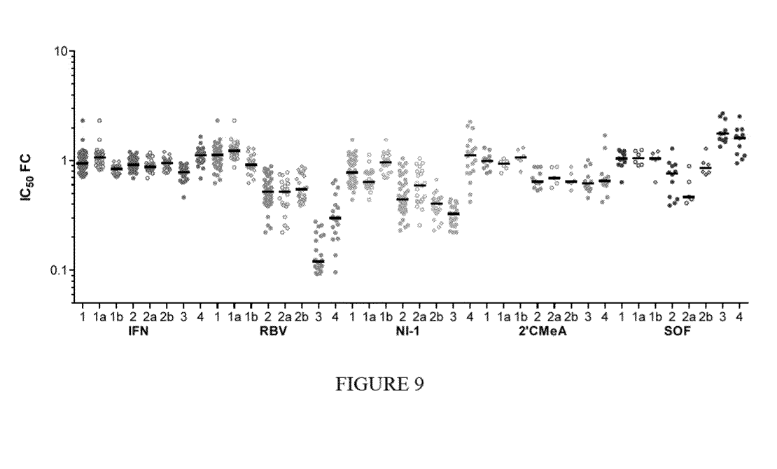

To determine the susceptibility of different genotype viruses to various inhibitors, the PhenoSense® HCV NS5B Assay was used. A panel of replicons were generated that contained patient-derived NS5B regions from GT1 (a/b), GT2 (a/b/k), GT3 (a/unknown), and GT4 (a/d/n/unknown) viruses. The various chimeric replicons were used in the phenotypic susceptibility assays described herein. Susceptibility to an interferon (IFN), ribavirin (RBV), nucleoside inhibitor-1 (NI-1), 2′C-methyl adenosine (2′CMeA), sofosbuvir (SOF) was tested to evaluate biological variation. The raw data is shown in FIG. 9 and analysis of the data in the table in FIG. 7, and the statistical significance is shown in FIG. 8. In FIG. 7, the inhibitor and genotype of the virus are indicated, as well as the number of viruses of the indicated genotype that were tested (“number of values”). The median, maximum, minimum, and range of IC₅₀ fold changes (compared to the IC₅₀ of reference virus) for each virus genotype for each inhibitor are shown. The range of IC₅₀ fold changes between all of the tested genotypes is shown at the bottom of the tables. FIG. 8 shows the significance of the variation in susceptibility to the inhibitors between the viruses of different genotypes as indicated, using a Wilcoxon rank sum test. The inhibitor is shown in the first column, and the genotypes of the two viruses that are being compared are shown in the second and third columns. The difference in susceptibilities (IC₅₀ fold change) between the two viruses that exhibited statistical significance shown in the fourth column.

The data used to generate the analysis in FIG. 7 is shown graphically in FIG. 9. Analysis of the data is shown in FIGS. 7 and 8, and the data is shown graphically in FIG. 8. This figure demonstrates the variation in susceptibility to IFN, RBV, NI-1, 2′CMeA, and SOF of viruses of different genotypes GT1 (a/b), GT2 (a/b/k), GT3 (a/unknown), and GT4 (a/d/n/unknown). The IC₅₀ fold change as compared to a reference virus value is plotted on the y axis, and the inhibitor and genotype of the virus tested are indicated on the x axis.

As shown above, GT1, GT2, GT3, and GT4 chimeric replicons had similar susceptibilities to IFN (left most eight groups in FIG. 9). GT1 chimeric replicons had similar susceptibilities to RBV, NI, 2′CmeA, and SOF. Overall, GT2, GT3, and GT4 chimeric replicons exhibited statistically significant increases (up to 15-fold) in susceptibilities to RBV, with replicons containing GT3 and GT4 NS5B being particularly susceptible. GT3 and GT4 chimeric replicons also showed significantly reduced SOF susceptibilities compared to GT1 chimeric replicons, while GT2 chimeric replicons had increased susceptibility (with GT2a chimeric replicons showing more increased susceptibility as compared to GT2b chimeric replicons (FIG. 8 and FIG. 9). Susceptibility to other nucleos(t)ide inhibitors (NIs) varied according to inhibitor and genotype with both GT2 and GT3 viruses exhibiting increased susceptibility to some NIs (FIGS. 6C, 6D, 8, and 9). Although the IFN susceptibilities are similar, there are small but significant differences as indicated in FIGS. 6C, 6D, and 8. Similarly, although the RBV and NI differences between GT1a and 1b viruses are similar, there are small but significant differences as indicated in FIGS. 6C, 6D, and 8.

RBV and SOF susceptibility was similar among replicons with GT1a/b NS5B, but was more variable between genotypes. A number of non-GT1 viruses exhibited relatively increased susceptibility to RBV. This observation may partially explain higher SVRs to RBV containing regimens among patients with some non-GT1 viruses. On whole, GT3 viruses exhibited reduced susceptibility to SOF compared to GT2, which may help to further explain differential SOF/RBV treatment responses between these genotypes. These data may contribute to improved SVR rates to RBV, NI, 2′CMeA, and SOF containing regimens, and combinations thereof, among patients infected with non-GT1 viruses. Therefore, this information would be useful to health care providers in determining the appropriate treatment regimen and/or treatment duration for a given individual. These data also may be useful to inform clinical trial design, pre-treatment decisions (e.g., drugs to use, number of drugs to combine, treatment duration), as well as for evaluating resistance. In particular, phenotypic data, in conjunction with clinical outcome data, may further strengthen the utility of the assay e.g. for developing clinical cut offs.

### Example 5

### Measurement of IC₅₀ FC and IC₉₅ FC to Detect Non-Nucleoside Inhibitor Susceptibility

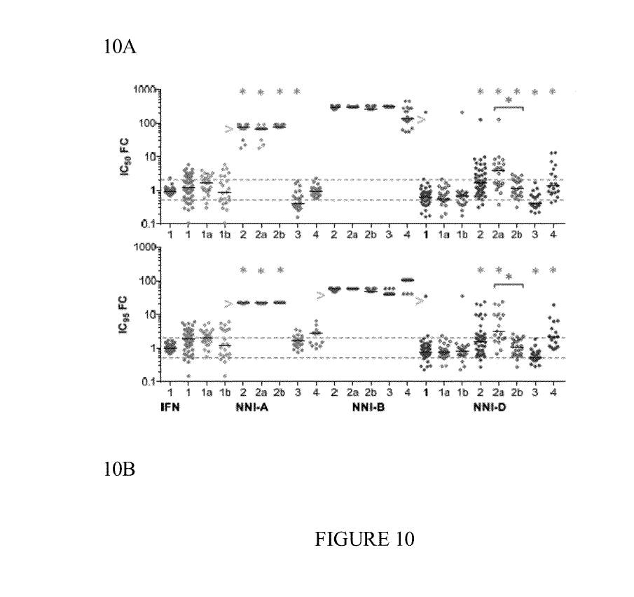

To determine the susceptibility of different genotype viruses to various non-nucleoside inhibitors, the PhenoSense® HCV NS5B Assay was used. A panel of replicons were generated that contained patient-derived NS5B regions from GT1, GT2, GT3, and GT4 viruses. The various chimeric replicons were used in the phenotypic susceptibility assays described herein. Susceptibility to IFN, NNI-A, NNI-B, and NNI-D was tested to evaluate biological variation. The results are shown in FIG. 10. The IC₅₀ fold change as compared to a reference Con1 virus value is plotted on the y axis in FIG. 10A, and the inhibitor and genotype of the virus tested are indicated on the x axis. Similarly, the IC₉₅ fold change as compared to a reference Con1 virus value is plotted on the y axis in FIG. 10B, and the inhibitor and genotype of the virus tested are indicated on the x axis.

The susceptibilities of the viruses of different genotypes to three different NNIs were more variable than that seen with other inhibitors. GT2 chimeric replicons showed significantly reduced susceptibilities to the NNI-A, NNI-B, and NNI-D inhibitors compared to GT1 chimeric replicons and/or the Con1 reference. GT3 chimeric replicons showed significantly reduced susceptibilities to the NNI-B inhibitor and increased susceptibilities to NNI-A and NNI-D inhibitors compared to GT1 chimeric replicons and/or the Con1 reference. GT4 chimeric replicons had reduced susceptibility to NNI-B and NNI-D inhibitors compared to GT1 chimeric replicons and/or the Con1 reference. NNI-D inhibitors can exhibit pan-genotypic activity among the tested genotypes as shown. These data would be useful to health care providers in determining the appropriate treatment regimen for a given individual. These data also may be useful to inform clinical trial design, pre-treatment decisions (e.g., drugs to use, number of drugs to combine, treatment duration), as well as for evaluating resistance. In particular, phenotypic data, in conjunction with clinical outcome data, may further strengthen the utility of the assay e.g. for developing clinical cut offs.

While the invention has been described and illustrated with reference to certain embodiments thereof those skilled in the art will appreciate that various changes, modifications and substitutions can be made therein without departing from the spirit and scope of the invention. All patents, published patent applications, and other non-patent references referred to herein are incorporated by reference in their entireties.

1. A method for determining the susceptibility of a hepatitis C virus (HCV) population to an HCV inhibitor, wherein the HCV inhibitor is ribavirin (RBV) or a nucleoside inhibitor (NI), comprising:
(a) introducing into a cell a resistance test vector comprising a patient derived segment from the HCV viral population, wherein the cell or the resistance test vector comprises an indicator nucleic acid that produces a detectable signal that is dependent on the HCV;
(b) measuring the expression of the indicator gene in the cell in the absence or presence of increasing concentrations of the HCV inhibitor;
(c) developing a standard curve of drug susceptibility for the HCV inhibitor, wherein the IC₅₀ fold change value, IC₉₅ fold change value, both, or the slope are detected in the standard curve;
(d) comparing the IC₅₀ fold change value, IC₉₅ fold change value, or both of the HCV population to IC₅₀ fold change value, IC₉₅ fold change value, or both for a control HCV population or comparing the slope of the standard curve of the HCV population to the slope of the standard curve for a control HCV population; and
(e) determining that the HCV population comprises HCV particles with an increased susceptibility to the HCV inhibitor when the IC₅₀ fold change value, IC₉₅ fold change value, or both are greater for the HCV population as compared to the IC₅₀ fold change value, IC₉₅ fold change value, or both for the control HCV population or determining that the HCV population comprises HCV particles with a reduced susceptibility to the HCV inhibitor when the slope of the standard curve of the HCV population is decreased as compared to the standard curve of the control population.

2. The method of claim 1, wherein the HCV inhibitor is a nucleoside inhibitor (NI).

3. The method of claim 1, wherein the HCV inhibitor is RBV.

4. The method of claim 1, wherein the control HCV population comprises Con1 HCV, H77 HCV, or the patient HCV population before treatment with the HCV inhibitor.

5. The method of claim 1, wherein the resistance test vector comprises the patient derived segment and the indicator gene.

6. The method of claim 1, wherein the patient derived segment comprises the NS5B region of the HCV.

7. The method of claim 1, wherein the indicator gene comprises a luciferase gene.

8. The method of claim 1, further comprising determining an appropriate treatment regimen for the patient based on the susceptibility determination of step (e).
